(19) United States
(12) Patent Application Publication
(10) Pub. No.: US 2024/0398882 A1
(43) Pub. Date: Dec. 5, 2024

(54) PAIN-RELIEF MEDICINE FOR HEADACHE

(71) Applicant: SEYED MOHAMMAD MOUSAVI MIRZAEI, BIRJAND (IR)

(72) Inventor: SEYED MOHAMMAD MOUSAVI MIRZAEI, BIRJAND (IR)

(21) Appl. No.: 18/800,030

(22) Filed: Aug. 10, 2024

Related U.S. Application Data
(63) Continuation-in-part of application No. PCT/IB2022/062831, filed on Dec. 28, 2022.
(60) Provisional application No. 63/308,606, filed on Feb. 10, 2022.

(51) Int. Cl. A61K 36/23; A61K 9/14; A61K 31/122; A61K 31/197; A61K 31/205; A61K 31/355; A61K 31/375; A61K 31/385; A61K 31/4188; A61K 31/4415; A61K 31/455; A61K 31/51; A61K 31/522; A61K 31/525; A61K 31/593; A61K 31/714; A61K 33/06; A61K 33/26; A61K 33/30; A61K 36/28; A61K 36/282; A61K 36/537; A61K 36/77; A61K 36/86; A61K 36/8905; A61K 36/899; A61K 36/9066

(52) U.S. Cl. CPC A61K 36/23; A61K 9/14; A61K 31/122; A61K 31/197; A61K 31/205; A61K 31/355; A61K 31/375; A61K 31/385; A61K 31/4188; A61K 31/4415; A61K 31/455; A61K 31/51; A61K 31/522; A61K 31/525; A61K 31/593; A61K 31/714; A61K 33/06; A61K 33/26; A61K 33/30; A61K 36/28; A61K 36/282; A61K 36/537; A61K 36/77; A61K 36/86; A61K 36/8905; A61K 36/899; A61K 36/9066

(57) ABSTRACT

A pain-relief medicine for healing headache is disclosed. The pain-relief medicine includes an herbal portion and a supplementary portion. The herbal portion includes a pimpinella anisum powder, an artemisia vulgaris powder, a viola odorata powder, a Cyperus species powder, a matricaria chamomilla powder, a salvia officinalis powder, an aesculus hippocastanum powder, a cymbopogon citratus powder, a trachyspermum ammi powder, and a curcuma longa powder. The supplementary portion includes coenzyme Q₁₀powder, carnitine powder, magnesium powder, α-lipoic acid powder, zinc powder, iron powder, vitamin C powder, vitamin E powder, vitamin D powder, vitamin B powder, and caffeine powder.

### RELATED APPLICATIONS

This application claims priority to and is a continuation-in-part of International Application Number PCT/IB2022/062831, filed on Dec. 28, 2022, entitled “A PAIN-RELIEF MEDICINE FOR HEALING HEADACHE”, which claims priority to U.S. Provisional Patent Application Ser. No. 63/308,606, filed on Feb. 10, 2022. International Application Number PCT/IB2022/062831 and U.S. Provisional Patent Application Ser. No. 63/308,606 are incorporated herein by reference in their entirety.

### TECHNICAL FIELD

The present disclosure generally relates to a pain-relief medicine, for example, a method of producing a pain-relief medicine for relieving headache.

### BACKGROUND ART

Pain is a signal of body for conveying a message that something in body is wrong. Nervous system which includes brain, spinal cord, and a complex network of nerves is responsible for alarming a damage to body. Pain is an enormous problem for human beings and can deteriorate quality of life. Pain that persist or recur longer than three months is called chronic pain which may exert economic burden on people. One of a known and frequent pain is headache which people may experience many times during their lives.

Headaches may happen due to different reasons including inflation, traction, muscle contraction, and vascular problems. Methods used for healing pain, and more particularly, headaches are home remedies and medical help. Home remedies may include using cold pack, dimming light, trying not to chew, using hot compress, hydrating, etc., which can partially lessen headaches. Medical help includes taking chemical drugs, herbal medication, etc., which can lessen or eliminate pain signs in a short period of time. However, chemical drugs have harmful side effects which may limit their usage.

Different strategies have been applied in prior art for healing headaches. For example, Hong Sun et al. presented a patent on “Methods for treating or preventing migraine headache” (U.S. Pat. No. 11,466,090B2). Hong Sun et al. used a method of administering anti-CGRP receptor or binding fragments to patient for healing migraine. Raphael Barkan et al. presented a patent on “Pharmaceutical compositions for headache, migraine, nausea and emesis” (U.S. Pat. No. 7,148,208B2). Raphael Barkan et al. used S-alkylsiothiouronium derivatives such as S-ethylisothiouronium diethylphosphate for healing headaches. The method is appropriate for treating migraines, nausea, vomiting, other medical conditions such as chemotherapy or radiotherapy, phonophobia and photophobia. Chemical medications used in these methods may cause harmful side effects on body.

There is, therefore, a need for a biocompatible, high efficiency medicine for treating headaches without harmful side effects. There is further a need for a cost effective and fast method to produce a pain-relief medicine for treating headaches.

### SUMMARY OF THE DISCLOSURE

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

According to one or more exemplary embodiments, the present disclosure is directed to a pain-relief medicine for healing headache. In an exemplary embodiment, an exemplary pain-relief medicine may include an herbal portion and a supplementary portion. In an exemplary embodiment, an exemplary pain-relief medicine may include an exemplary supplementary portion with a weight ratio to an exemplary herbal portion in a range of 1:7 to 7:1 (herbal portion:supplementary portion). In an exemplary embodiment, an exemplary herbal portion may include pimpinella anisum powder with a weight percentage in a range of 4 wt. % to 50 wt. % of a total amount of an exemplary herbal portion, artemisia vulgaris powder to an exemplary pimpinella anisum powder with a weight ratio in a range of 7:50 to 15:1 (artemisia vulgaris powder:pimpinella anisum powder), viola odorata powder to an exemplary pimpinella anisum powder with a weight ratio in a range of 2:25 to 25:2 (viola odorata powder:pimpinella anisum powder), Cyperus species powder to an exemplary pimpinella anisum powder with a weight ratio in a range of 1:50 to 15:2 (Cyperus species powder:pimpinella anisum powder), matricaria chamomilla powder to an exemplary pimpinella anisum powder with a weight ratio in a range of 1:50 to 15:4 (matricaria chamomilla powder:pimpinella anisum powder), salvia officinalis powder to an exemplary pimpinella anisum powder with a weight ratio in a range of 1:50 to 25:4 (salvia officinalis powder:pimpinella anisum powder), aesculus hippocastanum powder to an exemplary pimpinella anisum powder with a weight ratio in a range of 1:25 to 15:2 (aesculus hippocastanum powder:pimpinella anisum powder), cymbopogon citratus powder to an exemplary pimpinella anisum powder with a weight ratio in a range of 1:25 to 5:1 (cymbopogon citratus powder:pimpinella anisum powder), trachyspermum ammi powder to an exemplary pimpinella anisum powder with a weight ratio in a range of 1:50 to 5:1 (trachyspermum ammi powder:pimpinella anisum powder), and curcuma longa powder to an exemplary pimpinella anisum powder with a weight ratio in a range of 1:50 to 5:1 (curcuma longa powder:pimpinella anisum powder). In an exemplary embodiment, an exemplary supplementary portion may include coenzyme Q₁₀ powder to an exemplary pimpinella anisum powder with a weight ratio in a range of 50:1 to 1:10 (coenzyme Q₁₀:pimpinella anisum powder), carnitine powder to an exemplary pimpinella anisum powder with a weight ratio in a range of 75:1 to 3:5 (carnitine:pimpinella anisum powder), magnesium powder to an exemplary pimpinella anisum powder with a weight ratio in a range of 25:1 to 1:10 (magnesium:pimpinella anisum powder), α-lipoic acid powder to an exemplary pimpinella anisum powder with a weight ratio in a range of 75:1 to 1:5 (α-lipoic acid:pimpinella anisum powder), zinc powder to an exemplary pimpinella anisum powder with a weight ratio in a range of 10:1 to 1:50 (zinc:pimpinella anisum powder), iron powder to an exemplary pimpinella anisum powder with a weight ratio in a range of 10:1 to 1:25 (iron:pimpinella anisum powder), vitamin C powder to an exemplary pimpinella anisum powder with a weight ratio in a range of 125:1 to 1:5 (vitamin C:pimpinella anisum powder), vitamin E powder to an exemplary pimpinella anisum powder with a weight ratio in a range of 90:4 to 4.5:50 (vitamin E:pimpinella anisum powder), vitamin D powder to an exemplary pimpinella anisum powder with a weight ratio in a range of 0.0125:4 to 0.00125:50 (vitamin D:pimpinella anisum powder), vitamin B powder to an exemplary pimpinella anisum powder with a weight ratio in a range of 2.5:1 to 1:500 (vitamin B:pimpinella anisum powder), and caffeine powder to an exemplary pimpinella anisum powder with a weight ratio in a range of 25:1 to 1:50 (caffeine:pimpinella anisum powder).

In an exemplary embodiment, an exemplary pain-relief medicine may include at least one adhesive substance with a weight percentage of a total amount of an exemplary pain-relief medicine in a range of 2 wt. % to 10 wt. %. In an exemplary embodiment, an exemplary adhesive substance may include at least one of starch, polyvinylpyrrolidone (PVP), and combinations thereof. In an exemplary embodiment, an exemplary vitamin B may include at least one of B1 (thiamin), B2 (riboflavin), B3 (niacin), B5 (pantothenic acid), B6 (pyridoxine), B7 (biotin), B9 (folate [folic acid]), B12 (cobalamin), and combinations thereof. In an exemplary embodiment, an exemplary each of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include powder of at least one of root, stem, leaf, flower, fruit, and combinations thereof of an exemplary respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa.

In an exemplary embodiment, exemplary powder particles of each of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include particles with an average particle size in a range of 10 micrometer (μm) to 200 μm. In an exemplary embodiment, an exemplary pain-relief medicine may have a form of at least one of tablet, capsule, syrup, tea bag, suppository, effervescent tablet, and combinations thereof. In an exemplary embodiment, exemplary Cyperus species powder may include powder of at least one of Cyperus rotundus, Cyperus javanicus, Cyperus brevifolius, Cyperus kyllingia, Cyperus monocephalus, Cyperus compressus, Cyperus articulatus, Cyperus pedunculatus, Cyperus nitidus, Cyperus sexangularis, Cyperus kilimandscharicus, Cyperus latifolius, Cyperus maculatus, Cyperus natalensis, Cyperus erectus, Cyperus mundii, Cyperus esculentus, Cyperus flavescens, and combinations thereof.

In an exemplary embodiment, an exemplary powder particles of each of an exemplary coenzyme Q₁₀ powder, an exemplary carnitine powder, an exemplary magnesium powder, an exemplary α-lipoic acid powder, an exemplary zinc powder, an exemplary iron powder, an exemplary vitamin C powder, an exemplary vitamin E powder, an exemplary vitamin D powder, an exemplary vitamin B powder, and an exemplary caffeine powder may include particles with an average particle size in a range of 10 μm to 200 μm.

According to one or more exemplary embodiments, the present disclosure is directed to a method for producing a pain-relief medicine. In an exemplary embodiment, an exemplary method may include forming an herbal portion, forming a supplementary portion by mixing powders of a plurality of supplementary compounds, and forming an herbal-supplementary mixture by mixing the herbal portion and the supplementary portion. In an exemplary embodiment, an exemplary plurality of supplementary compounds may include at least one of coenzyme Q₁₀ powder, carnitine powder, magnesium powder, α-lipoic acid powder, zinc powder, iron powder, vitamin C powder, vitamin E powder, vitamin D powder, vitamin B powder, caffeine powder, and combinations thereof. In an exemplary embodiment, forming an exemplary herbal portion may include forming an herbal mixture by mixing powders of a plurality of herbal compounds, forming an herbal suspension by mixing an exemplary herbal mixture with a hydroalcoholic solution, and isolating an exemplary herbal portion. In an exemplary embodiment, an exemplary herbal portion may include an aqueous extract of an exemplary herbal mixture by evaporating an exemplary alcohol portion of an exemplary herbal suspension. In an exemplary embodiment, exemplary powders of an exemplary plurality of herbal compounds may include artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder. In an exemplary embodiment, an exemplary hydroalcoholic solution may include an alcohol portion and a water portion.

In an exemplary embodiment, an exemplary method may further include forming exemplary powders of an exemplary plurality of the supplementary compounds by grinding each respective supplementary compound to particles. In an exemplary embodiment, each particle may have an average particle size in a range of 1 μm to 200 μm.

In an exemplary embodiment, forming an exemplary herbal suspension may include mixing an exemplary herbal mixture with an exemplary hydroalcoholic solution using a mixer with a stirring speed in a range of 50 revolutions per minute (rpm) to 200 rpm.

In an exemplary embodiment, forming an exemplary herbal suspension may include mixing an exemplary herbal mixture with an exemplary hydroalcoholic solution using a mixer for a time period in a range of 5 minutes to 10 minutes.

In an exemplary embodiment, forming an exemplary herbal-supplementary mixture may include mixing an exemplary herbal portion and an exemplary supplementary portion in a mixer with a stirring speed in a range of 50 rpm to 200 rpm.

In an exemplary embodiment, forming an exemplary herbal-supplementary mixture may include mixing an exemplary herbal portion and an exemplary supplementary portion in a mixer for a time period in a range of 5 minutes to 10 minutes.

According to one or more exemplary embodiments, the present disclosure is directed to a method for producing a pain-relief medicine in a form of tablet. In an exemplary embodiment, an exemplary method may include forming an herbal portion, forming a supplementary portion by mixing powders of a plurality of supplementary compounds, the plurality of supplementary compounds, forming a tablet mixture by mixing the herbal portion with the supplementary portion, applying a pressure on the tablet mixture in a range of 2500 (Kilopascal) kPa to 6500 kPa. In an exemplary embodiment, forming an exemplary herbal portion may include forming an herbal mixture by mixing powders of a plurality of herbal compounds, the powders of the plurality of herbal compounds, forming an herbal suspension by mixing the herbal mixture with a hydroalcoholic solution, isolating the herbal portion comprising an aqueous-herbal extract of the herbal mixture by evaporating the alcohol portion of the herbal suspension, and forming an extract powder of the herbal mixture by freeze-drying the isolated aqueous-herbal extract of the herbal mixture. In an exemplary embodiment, exemplary powders of an exemplary plurality of herbal compounds may include artemisia vulgaris powder, viola odorata powder, Cyperus species. powder, matricaria chamomilla powder, salvia officinalis powder, aesculus hippocastanum powder, cymbopogon citratus powder, trachyspermum ammi powder, and curcuma longa powder. In an exemplary embodiment, exemplary hydroalcoholic solution may include an alcohol portion and a water portion. In an exemplary embodiment, exemplary plurality of supplementary compounds may include at least one of coenzyme Q₁₀ powder, carnitine powder, magnesium powder, α-lipoic acid powder, zinc powder, iron powder, vitamin C powder, vitamin E powder, vitamin D powder, vitamin B powder, caffeine powder, and combinations thereof.

In an exemplary embodiment, an exemplary method may further include forming adhesive bonds among particles of an exemplary herbal-supplementary mixture by mixing at least one adhesive substance with an exemplary herbal-supplementary mixture. In an exemplary embodiment, an exemplary at least one adhesive substance may include at least one of starch, polyvinylpyrrolidone (PVP), and combinations thereof.

In an exemplary embodiment, forming an exemplary adhesive bonds among particles of an exemplary herbal-supplementary mixture may include mixing an exemplary at least one adhesive substance with an exemplary herbal-supplementary mixture in a mixer with a stirring speed in a range of 50 rpm to 200 rpm.

In an exemplary embodiment, forming an exemplary adhesive bonds among particles of an exemplary herbal-supplementary mixture may include mixing an exemplary at least one adhesive substance with an exemplary herbal-supplementary mixture in a mixer for a time period in a range of 5 minutes to 10 minutes.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a flowchart of a method for producing a pain-relief medicine for healing headache, consistent with one or more exemplary embodiments of the present disclosure.

FIG. 1B illustrates a flowchart of a method of forming an exemplary herbal portion, consistent with one or more exemplary embodiments of the present disclosure.

FIG. 2A illustrates a flowchart of a method for producing a pain-relief medicine in a form of tablet for healing headache, consistent with one or more exemplary embodiments of the present disclosure.

FIG. 2B illustrates a flowchart of a method for producing an exemplary herbal portion of a pain-relief medicine in a form of tablet, consistent with one or more exemplary embodiments of the present disclosure.

FIGS. 3A-3V illustrate an example scenario of producing an herbal portion.

FIGS. 4A-4B illustrate an example scenario of producing a pain relief medicine.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The novel features which are believed to be characteristic of the present disclosure, as to its structure, organization, use and method of operation, together with further objectives and advantages thereof, will be better understood from the following discussion. In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

The present disclosure is directed to exemplary embodiments of a pain-relief medicine and a method to produce an exemplary pain-relief medicine. In an exemplary embodiment, an exemplary pain-relief medicine may have a form of at least one of tablet, capsule, syrup, tea bag, suppository, effervescent tablet, and combinations thereof. In an exemplary embodiment, an exemplary pain-relief medicine may be used for relieving headache. In an exemplary embodiment, an exemplary headache may include at least one of nervous headaches, migraine headaches, headaches due to head and neck problems, headaches caused by joint problems, chronic daily headaches, headaches due to increased brain pressure, head trauma, headaches due to spasms, and combinations thereof. In an exemplary embodiment, an exemplary painrelief medicine may also relieve at least one of sleep disorder, menstrual problems, digestive problems, mental problems, hormonal problems, increased brain pressure, and combinations thereof. In an exemplary embodiment, an exemplary pain-relief medicine may include an herbal portion and a supplementary portion. In an exemplary embodiment, an exemplary pain-relief medicine may include an exemplary herbal portion and an exemplary supplementary portion with a weight ratio in a range of 1:7 to 7:1 (herbal portion:supplementary portion).

In an exemplary embodiment, an exemplary herbal portion may include pimpinella anisum powder, artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder. In an exemplary embodiment, each of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include powder of at least one of root, stem, leaf, flower, fruit, and combinations thereof of an exemplary respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa. In an exemplary embodiment, each powder particle of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include particles with an average size in a range of 10 μm to 200 μm. In an exemplary embodiment, exemplary Cyperus species powder may include powder of at least one of Cyperus rotundus, Cyperus javanicus, Cyperus brevifolius, Cyperus kyllingia, Cyperus monocephalus, Cyperus compressus, Cyperus articulatus, Cyperus pedunculatus, Cyperus nitidus, Cyperus sexangularis, Cyperus kilimandscharicus, Cyperus latifolius, Cyperus maculatus, Cyperus natalensis, Cyperus erectus, Cyperus mundii, Cyperus esculentus, Cyperus flavescens, and combinations thereof.

In an exemplary embodiment, an exemplary pain-relief medicine may include an exemplary pimpinella anisum powder with a weight percentage in a range of 4 wt. % to 50 wt. % of a total amount of an exemplary herbal portion. In an exemplary embodiment, an exemplary pain-relief medicine may include an exemplary artemisia vulgaris powder with a weight ratio to an exemplary pimpinella anisum powder in a range of 7:50 to 15:1 (artemisia vulgaris powder:pimpinella anisum powder). In an exemplary embodiment, an exemplary pain-relief medicine may include an exemplary viola odorata powder with a weight ratio to an exemplary pimpinella anisum powder in a range of 2:25 to 25:2 (viola odorata powder:pimpinella anisum powder). In an exemplary embodiment, an exemplary pain-relief medicine may include an exemplary Cyperus species powder with a weight ratio to an exemplary pimpinella anisum powder in a range of 1:50 to 15:2 (Cyperus species powder:pimpinella anisum powder). In an exemplary embodiment, an exemplary pain-relief medicine may include an exemplary matricaria chamomilla powder with a weight ratio to an exemplary pimpinella anisum powder in a range of 1:50 to 15:4 (matricaria chamomilla powder:pimpinella anisum powder). In an exemplary embodiment, an exemplary pain-relief medicine may include an exemplary salvia officinalis powder with a weight ratio to an exemplary pimpinella anisum powder in a range of 1:50 to 25:4 (salvia officinalis powder:pimpinella anisum powder). In an exemplary embodiment, an exemplary pain-relief medicine may include an exemplary aesculus hippocastanum powder with a weight ratio to an exemplary pimpinella anisum powder in a range of 1:25 to 15:2 (aesculus hippocastanum powder:pimpinella anisum powder). In an exemplary embodiment, an exemplary pain-relief medicine may include an exemplary cymbopogon citratus powder with a weight ratio to an exemplary pimpinella anisum powder in a range of 1:25 to 5:1 (cymbopogon citratus powder:pimpinella anisum powder). In an exemplary embodiment, an exemplary pain-relief medicine may include an exemplary trachyspermum ammi powder with a weight ratio to an exemplary pimpinella anisum powder in a range of 1:50 to 5:1 (trachyspermum ammi powder:pimpinella anisum powder). In an exemplary embodiment, an exemplary pain-relief medicine may include an exemplary curcuma longa powder with a weight ratio to an exemplary pimpinella anisum powder in a range of 1:50 to 5:1 (curcuma longa powder:pimpinella anisum powder).

In an exemplary embodiment, an exemplary supplementary portion may include coenzyme Q₁₀ powder, carnitine powder, magnesium powder, α-lipoic acid powder, zinc powder, iron powder, vitamin C powder, vitamin E powder, vitamin D powder, vitamin B powder, and caffeine powder. In an exemplary embodiment, powder particles of each of an exemplary coenzyme Q₁₀ powder, an exemplary carnitine powder, an exemplary magnesium powder, an exemplary α-lipoic acid powder, an exemplary zinc powder, an exemplary iron powder, an exemplary vitamin C powder, an exemplary vitamin E powder, an exemplary vitamin D powder, an exemplary vitamin B powder, and an exemplary caffeine powder may include particles with an average particle size in a range of 1 μm to 200 μm. In an exemplary embodiment, an exemplary pain-relief medicine may include an exemplary coenzyme Q₁₀ powder with a weight ratio to an exemplary pimpinella anisum powder in a range of 50:1 to 1:10 (coenzyme Q10 powder:pimpinella anisum powder). In an exemplary embodiment, an exemplary pain-relief medicine may include an exemplary carnitine powder with a weight ratio to an exemplary pimpinella anisum powder in a range of 75:1 to 3:5 (carnitine powder:pimpinella anisum powder). In an exemplary embodiment, an exemplary pain-relief medicine may include an exemplary magnesium powder with a weight ratio to an exemplary pimpinella anisum powder in a range of 25:1 to 1:10 (magnesium powder:pimpinella anisum powder). In an exemplary embodiment, an exemplary pain-relief medicine may include an exemplary α-lipoic acid powder with a weight ratio to an exemplary pimpinella anisum powder in a range of 75:1 to 1:5 (α-lipoic acid:pimpinella anisum powder). In an exemplary embodiment, an exemplary pain-relief medicine may include an exemplary zinc powder with a weight ratio to an exemplary pimpinella anisum powder in a range of 10:1 to 1:50 (zinc powder:pimpinella anisum powder). In an exemplary embodiment, an exemplary pain-relief medicine may include an exemplary iron powder with a weight ratio to an exemplary pimpinella anisum powder in a range of 10:1 to 1:25 (iron powder:pimpinella anisum powder). In an exemplary embodiment, an exemplary pain-relief medicine may include an exemplary vitamin C powder with a weight ratio to an exemplary pimpinella anisum powder in a range of 125:1 to 1:5 (vitamin C powder:pimpinella anisum powder). In an exemplary embodiment, an exemplary pain-relief medicine may include an exemplary vitamin E powder with a weight ratio to an exemplary pimpinella anisum powder in a range of 90:4 to 4.5:50 (vitamin E powder:pimpinella anisum powder). In an exemplary embodiment, an exemplary pain-relief medicine may include an exemplary vitamin D powder with a weight ratio to an exemplary pimpinella anisum powder in a range of 0.0125:4 to 0.00125:50(vitamin D powder:pimpinella anisum powder). In an exemplary embodiment, an exemplary pain-relief medicine may include an exemplary vitamin B powder with a weight ratio to an exemplary pimpinella anisum powder in a range of 2.5:1 to 1:500 (vitamin B powder:pimpinella anisum powder). In an exemplary embodiment, an exemplary pain-relief medicine may include an exemplary caffeine powder with a weight ratio to an exemplary pimpinella anisum powder in a range of 25:1 to 1:50 (caffeine powder:pimpinella anisum powder). In an exemplary embodiment, an exemplary vitamin B may include at least one of B1 (thiamin), B2 (riboflavin), B3 (niacin), B5 (pantothenic acid), B6 (pyridoxine), B7 (biotin), B9 (folate [folic acid]), B12 (cobalamin), and combinations thereof. In an exemplary embodiment, an exemplary pain-relief medicine may further include at least one adhesive substance. In an exemplary embodiment, an exemplary adhesive substance may be used to form adhesive bonds among particles of an exemplary herbal portion and particles of an exemplary supplementary portion. In an exemplary embodiment, an exemplary adhesive substance may be used to produce an exemplary pain-relief medicine in a form of at least one of tablet, capsule, suppository, effervescent tablet, and combinations thereof. In an exemplary embodiment, an exemplary adhesive substance may include at least one of starch, polyvinylpyrrolidone (PVP), and combinations thereof. In an exemplary embodiment, an exemplary pain-relief medicine may include an exemplary at least one adhesive substance with a weight percentage of a total amount of an exemplary pain-relief medicine in a range of 2 wt. % to 10 wt. %. In an exemplary embodiment, a predetermined amount of an exemplary pain-relief medicine may be taken per day. In an exemplary embodiment, an exemplary predetermined amount may include a weight in a range of 4 grams (gr) to 8 gr of an exemplary pain-relief medicine per day.

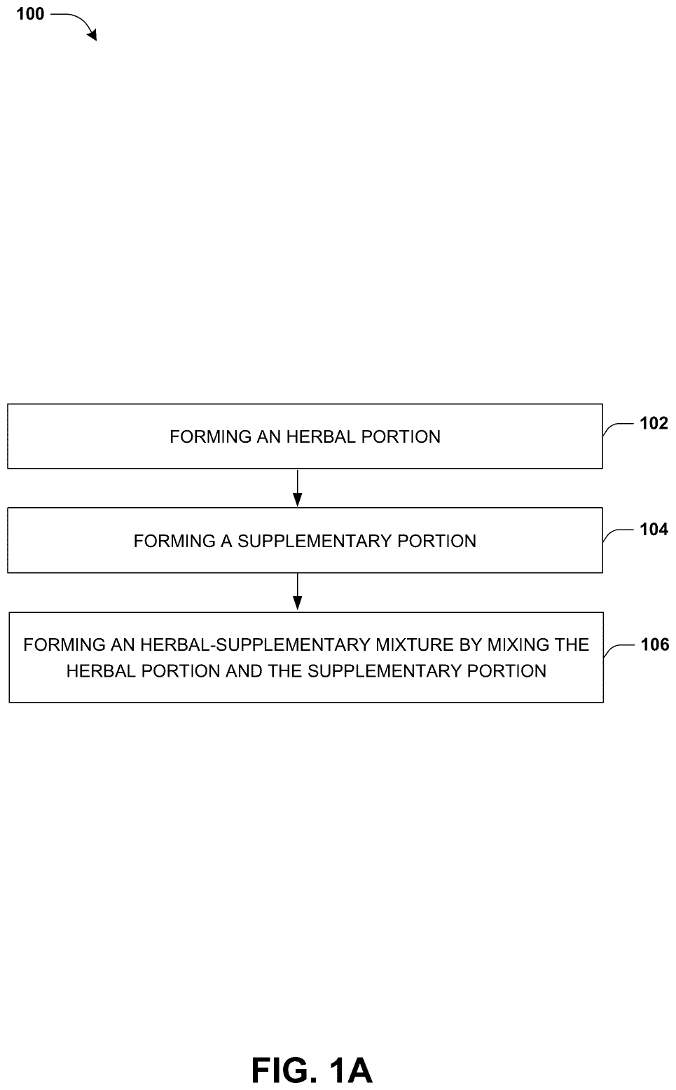

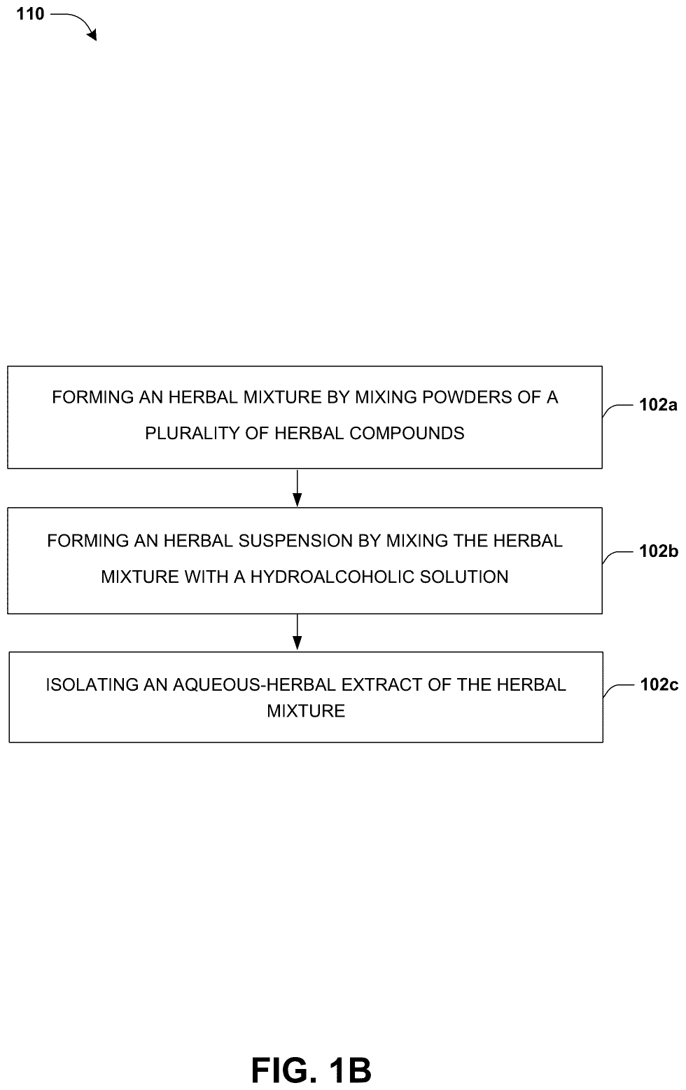

FIG. 1A illustrates a flowchart of a method 100 for producing a pain-relief medicine for healing headache, consistent with one or more exemplary embodiments of the present disclosure. In an exemplary embodiment, method 100 may include a step 102 of forming an herbal portion, a step 104 of forming a supplementary portion, and a step 106 of forming an herbal-supplementary mixture by mixing the herbal portion and the supplementary portion.

In further detail with respect to step 102, step 102 of forming an herbal portion may be illustrated using FIG. 1B. FIG. 1B illustrates a flowchart of a method 110 of forming an exemplary herbal portion, consistent with one or more exemplary embodiments of the present disclosure. In an exemplary embodiment, method 110 may include a step 102a of forming an herbal mixture by mixing powders of a plurality of herbal compounds, a step 102b of forming an herbal suspension by mixing the herbal mixture with a hydroalcoholic solution, and a step 102c of isolating an aqueous-herbal extract of the herbal mixture.

In further detail with respect to step 102a, step 102a of forming an herbal mixture may include mixing a plurality of herbal powders in a mixer. In an exemplary embodiment, an exemplary plurality of herbal powders may be produced by washing a plurality of herbal materials, drying an exemplary plurality of herbal materials, and grinding an exemplary plurality of herbal materials. In an exemplary embodiment, an exemplary plurality of herbal materials may be washed by dipping an exemplary plurality of herbal materials in water for a time period in a range of 5 minutes to 10 minutes. In an exemplary embodiment, an exemplary plurality of herbal materials may be mixed with water with a weight ratio in range of 1:2 to 1:10 (an exemplary plurality of herbal materials:water). In an exemplary embodiment, an exemplary plurality of herbal materials may be mixed with water utilizing a mixer with a stirring speed in a range of 50 rpm to 200 rpm. In an exemplary embodiment, an exemplary plurality of herbal materials may be mixed with water for a time period in a range of 5 minutes to 10 minutes. In an exemplary embodiment, an exemplary plurality of herbal materials may be dried after an exemplary washing process. In an exemplary embodiment, an exemplary plurality of herbal materials may be heated at a temperature in a range of 20 Degrees Celsius (° C.) to 60° C. In an exemplary embodiment, an exemplary plurality of herbal materials may be heated for a time period in a range of 1 day to 5 days. In an exemplary embodiment, after drying an exemplary plurality of herbal material, an exemplary plurality of herbal materials may be grinded to form an exemplary plurality of herbal powders. In an exemplary embodiment, an exemplary plurality of herbal powders may be sieved for excluding particles larger than 200 μm. In an exemplary embodiment, an exemplary plurality of herbal powders may include particles with an average particle size in a range of 10 μm to 200 μm. In an exemplary embodiment, an exemplary herbal mixture may be produced by mixing an exemplary plurality of herbal powders in a mixer. In an exemplary embodiment, an exemplary plurality of herbal powders may be mixed in an exemplary mixer with a stirring speed in a range of 50 rpm to 200 rpm. In an exemplary embodiment, an exemplary plurality of herbal powders may be mixed in an exemplary mixer for a time period in a range of 5 minutes to 10 minutes. In an exemplary embodiment, an exemplary plurality of herbal powders may include pimpinella anisum powder, artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder. In an exemplary embodiment, each powder particle of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include an average particle size in a range of 10 μm to 200 μm. In an exemplary embodiment, an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include powder of at least one of root, stem, leaf, flower, fruit, and combinations thereof of an exemplary respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa. In an exemplary embodiment, an exemplary pimpinella anisum powder may include a weight percentage in a range of 4 wt. % to 50 wt. % of a total amount of an exemplary herbal portion. In an exemplary embodiment, an exemplary pain-relief medicine may include an exemplary artemisia vulgaris powder with a weight ratio to an exemplary pimpinella anisum powder in a range of 7:50 to 15:1 (artemisia vulgaris powder:pimpinella anisum powder). In an exemplary embodiment, an exemplary pain-relief medicine may include an exemplary viola odorata powder with a weight ratio to an exemplary pimpinella anisum powder in a range of 2:25 to 25:2 (viola odorata powder:pimpinella anisum powder). In an exemplary embodiment, an exemplary pain-relief medicine may include an exemplary Cyperus species powder with a weight ratio to an exemplary pimpinella anisum powder in a range of 1:50 to 15:2 (Cyperus species powder:pimpinella anisum powder). In an exemplary embodiment, an exemplary pain-relief medicine may include an exemplary matricaria chamomilla powder with a weight ratio to an exemplary pimpinella anisum powder in a range of 1:50 to 15:4 (matricaria chamomilla powder:pimpinella anisum powder). In an exemplary embodiment, an exemplary pain-relief medicine may include an exemplary salvia officinalis powder with a weight ratio to an exemplary pimpinella anisum powder in a range of 1:50 to 25:4 (salvia officinalis powder:pimpinella anisum powder). In an exemplary embodiment, an exemplary pain-relief medicine may include an exemplary aesculus hippocastanum powder with a weight ratio to an exemplary pimpinella anisum powder in a range of 1:25 to 15:2 (aesculus hippocastanum powder:pimpinella anisum powder). In an exemplary embodiment, an exemplary pain-relief medicine may include an exemplary cymbopogon citratus powder with a weight ratio to an exemplary pimpinella anisum powder in a range of 1:25 to 5:1 (cymbopogon citratus powder:pimpinella anisum powder). In an exemplary embodiment, an exemplary pain-relief medicine may include an exemplary trachyspermum ammi powder with a weight ratio to an exemplary pimpinella anisum powder in a range of 1:50 to 5:1 (trachyspermum ammi powder:pimpinella anisum powder). In an exemplary embodiment, an exemplary pain-relief medicine may include an exemplary curcuma longa powder with a weight ratio to an exemplary pimpinella anisum powder in a range of 1:50 to 5:1 (curcuma longa powder:pimpinella anisum powder).

In further detail with respect to step 102b, step 102b of forming an herbal suspension may include mixing an exemplary herbal mixture with a hydroalcoholic solution. In an exemplary embodiment, an exemplary herbal mixture may be mixed with an exemplary hydroalcoholic solution using a mixer with a stirring speed in a range of 50 rpm to 200 rpm. In an exemplary embodiment, an exemplary herbal mixture may be mixed with an exemplary hydroalcoholic solution at a temperature in a range of 15° C. to 60° C. In an exemplary embodiment, an exemplary herbal mixture may be mixed with an exemplary hydroalcoholic solution for a time period in a range of 30 minutes to 72 hours. In an exemplary embodiment, an exemplary hydroalcoholic solution may include an alcoholic portion and water. In an exemplary embodiment, an exemplary alcoholic portion may include at least one of ethanol, and combinations thereof. In an exemplary embodiment, an exemplary hydroalcoholic solution may include an exemplary alcoholic portion with a weight ratio to water in a range of 5:95 to 99:1 (alcoholic portion:water). In an exemplary embodiment, exemplary water may include distilled water. In an exemplary embodiment, an exemplary hydroalcoholic solution may be mixed with an exemplary herbal mixture with a weight ratio in a range of 5:1 to 100:1 (hydroalcoholic solution:herbal mixture). In an exemplary embodiment, an extract of an exemplary herbal mixture may be dissolved in an exemplary hydroalcoholic solution by mixing an exemplary herbal mixture with an exemplary hydroalcoholic solution.

In further detail with respect to step 102c, step 102c may include isolating an aqueous herbal extract of an exemplary herbal mixture. In an exemplary embodiment, an exemplary aqueous herbal extract of an exemplary herbal mixture may be isolated from an exemplary herbal mixture as an exemplary herbal portion. In an exemplary embodiment, isolating an exemplary herbal portion (or an exemplary aqueous herbal extract of an exemplary herbal mixture) may include filtering an exemplary mixture of an exemplary hydroalcoholic solution and an exemplary herbal mixture. In an exemplary embodiment, an exemplary herbal mixture may be separated from an exemplary hydroalcoholic solution using a filter with pore size in a range of 1 μm to 30 μm. In an exemplary embodiment, an exemplary hydroalcoholic solution may contain dissolved extract of an exemplary herbal mixture therein. In an exemplary embodiment, an exemplary alcoholic portion of an exemplary hydroalcoholic solution may be separated from water. In an exemplary embodiment, an exemplary alcoholic portion of an exemplary hydroalcoholic solution may be separated from water using a rotary evaporator device. As used herein an exemplary rotary evaporator device may refer to a device that remove solvent from samples by evaporation. In an exemplary embodiment, an exemplary hydroalcoholic solution may be heated at a temperature in a range of 15° C. to 60° C. using a heater. In an exemplary embodiment, an exemplary mixture an exemplary hydroalcoholic solution and an exemplary herbal mixture may be heated for a time period in a range of 30 minutes to 72 hours. In an exemplary embodiment, water may contain an exemplary extract of an exemplary herbal mixture. In an exemplary embodiment, an exemplary extract of an exemplary herbal mixture may be dissolved in water to form an aqueous solution of an exemplary herbal extract of an exemplary herbal mixture. In an exemplary embodiment, an exemplary aqueous solution of an exemplary herbal extract may be used as an exemplary herbal portion for producing an exemplary pain-relief medicine. In an exemplary embodiment, an exemplary aqueous solution of an exemplary herbal extract may be used to form an exemplary pain-relief medicine in a form of syrup.

Referring back to FIG. 1A, in further detail with respect to step 104, step 104 of forming a supplementary portion may include forming a plurality of supplementary powders of a plurality of supplementary materials. In an exemplary embodiment, an exemplary plurality of supplementary powders may be produced by grinding a plurality of supplementary materials to an average particle size in a range of 1 μm to 200 μm. In an exemplary embodiment, an exemplary plurality of supplementary powders may be mixed in a mixer with a stirring speed in a range of 50 rpm to 200 rpm. In an exemplary embodiment, an exemplary plurality of supplementary powders may be mixed together in a mixer for a time period in a range of 5 minutes to 10 minutes. In an exemplary embodiment, an exemplary plurality of supplementary powders may include coenzyme Q₁₀ powder, carnitine powder, magnesium powder, α-lipoic acid powder, zinc powder, iron powder, vitamin C powder, vitamin E powder, vitamin D powder, vitamin B powder, and caffeine powder. In an exemplary embodiment, exemplary plurality of supplementary powders may include particles with an average particle size in a range of 1 μm to 200 μm. In an exemplary embodiment, an exemplary vitamin B may include at least one of thiamin (B1), riboflavin (B2), niacin (B3), pantothenic acid (B5), pyridoxine (B6), biotin (B7), folate [folic acid] (B9), cobalamin (B12), and combinations thereof.

In further detail with respect to step 106, step 106 of forming an herbal-supplementary mixture may include mixing an exemplary herbal portion and an exemplary supplementary portion. In an exemplary embodiment, an exemplary herbal portion may be mixed with an exemplary supplementary portion in a mixer with a stirring speed in a range of 50 rpm to 200 rpm. In an exemplary embodiment, an exemplary herbal portion may be mixed with an exemplary supplementary portion for a time period in a range of 5 minutes to 10 minutes. In an exemplary embodiment, an exemplary herbal portion may be mixed with an exemplary supplementary portion with a weight ratio in a range of 7:1 to 1:7 (supplementary portion:herbal portion). In an exemplary embodiment, an exemplary aqueous solution of an exemplary herbal extract may be mixed with an exemplary supplementary portion. In an exemplary embodiment, an exemplary herbal-supplementary mixture may be used for producing an exemplary pain-relief medicine in a form of syrup.

In an exemplary embodiment, an exemplary pain-relief medicine may further include at least one adhesive substance for producing an exemplary pain-relief medicine in a form of at least one of tablet, capsule, tea bag, suppository, effervescent tablet, and combinations thereof. In an exemplary embodiment, for producing an exemplary pain-relief medicine in a form of at least one of tablet, capsule, tea bag, suppository, effervescent tablet, and combinations thereof, an exemplary aqueous-herbal extract may be dried utilizing a freeze dryer. In an exemplary embodiment, an exemplary aqueous-herbal extract of an exemplary herbal mixture may be freeze dried utilizing an exemplary freeze dryer for a time period in a range of 24 hours to 72 hours. In an exemplary embodiment, an exemplary aqueous-herbal extract of an exemplary herbal mixture may be freeze dried utilizing an exemplary freeze dryer at a temperature in a range of −40° C. to −60° C. In an exemplary embodiment, a powdered-herbal extract of an exemplary herbal mixture may be produced by freeze drying an exemplary aqueous-herbal extract of an exemplary herbal mixture. In an exemplary embodiment, at least one adhesive substance may be mixed with an exemplary powdered-herbal extract of an exemplary herbal mixture and an exemplary supplementary portion. In an exemplary embodiment, an exemplary adhesive substance may form connections among exemplary particles of an exemplary powdered-herbal extract of an exemplary herbal mixture and an exemplary supplementary portion. In an exemplary embodiment, an exemplary powdered-herbal extract of an exemplary herbal mixture, an exemplary supplementary portion, and an exemplary at least one adhesive substance may be mixed together in a mixer with a stirring speed in a range of 50 rpm to 200 rpm. In an exemplary embodiment, an exemplary powdered-herbal extract of an exemplary herbal mixture, an exemplary supplementary portion, and an exemplary at least one adhesive substance may be mixed together in a mixer for a time period in a range of 5 minutes to 10 minutes. In an exemplary embodiment, an exemplary at least one adhesive substance may include at least one of starch, PVP, and combinations thereof. In an exemplary embodiment, an exemplary pain-relief medicine may include an exemplary at least one adhesive substance with a weight percentage of a total amount of an exemplary pain-relief medicine in a range of 2 wt. % to 10 wt. %.

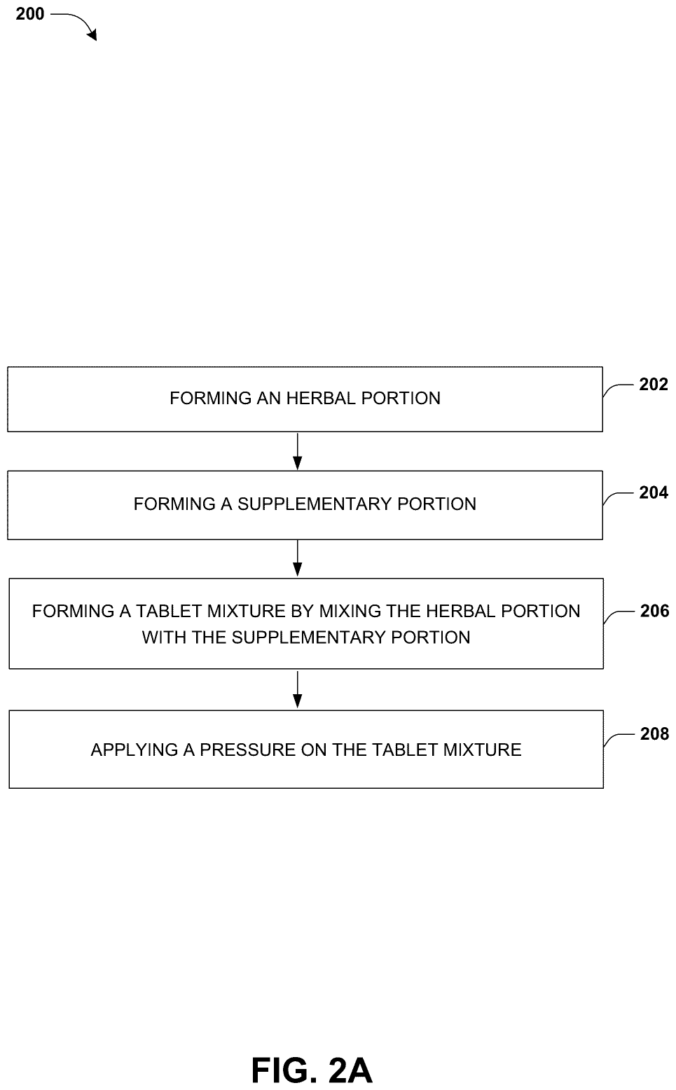

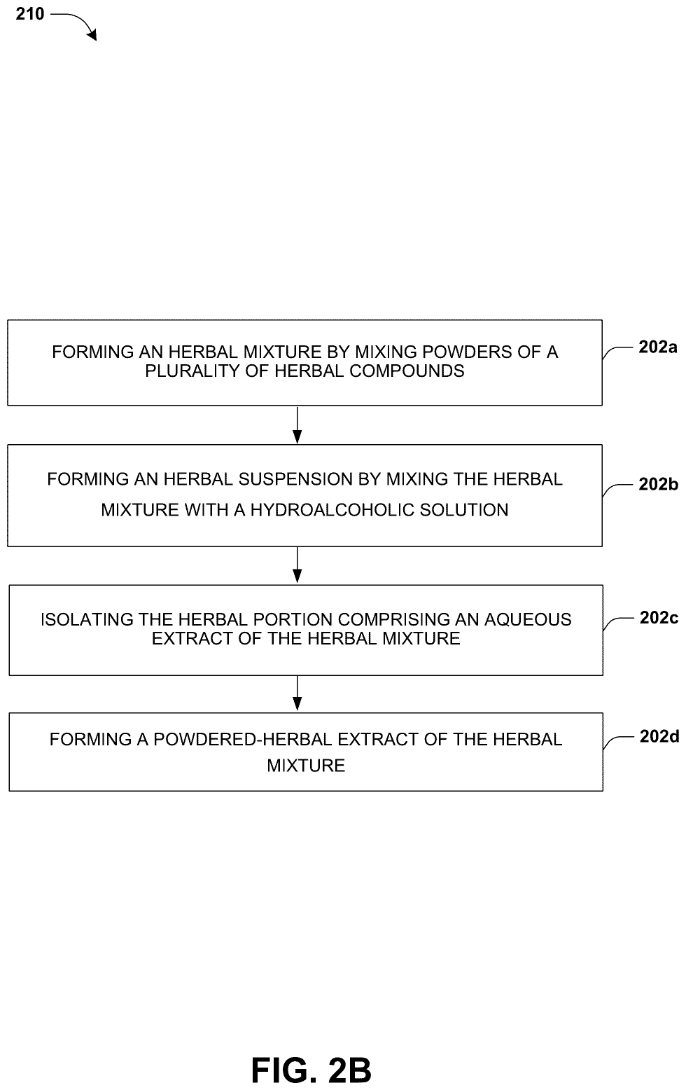

FIG. 2A illustrates a flowchart of a method 200 for producing a pain-relief medicine in a form of tablet for healing headache, consistent with one or more exemplary embodiments of the present disclosure. In an exemplary embodiment, method 200 may include a step 202 of forming an herbal portion, a step 204 of forming a supplementary portion, a step 206 of forming a tablet mixture by mixing the herbal portion with the supplementary portion, and a step 208 of applying a pressure on the tablet mixture.

In further detail with respect to step 202, step 202 of forming an herbal portion may be illustrated using FIG. 2B. FIG. 2B illustrates a flowchart of a method 210 for producing an exemplary herbal portion of a pain-relief medicine in a form of tablet, consistent with one or more exemplary embodiments of the present disclosure. In an exemplary embodiment, method 210 may include a step 202a of forming an herbal mixture by mixing powders of a plurality of herbal compounds, step 202b of forming an herbal suspension by mixing the herbal mixture with a hydroalcoholic solution, step 202c of isolating the herbal portion comprising an aqueous extract of the herbal mixture, and step 202d of forming a powdered-herbal extract of the herbal mixture.

In further detail with respect to step 202a, step 202a of forming an herbal mixture may include mixing a plurality of herbal powders in a mixer. In an exemplary embodiment, an exemplary plurality of herbal powders may be produced by washing a plurality of herbal materials. In an exemplary embodiment, an exemplary plurality of herbal materials may be washed by dipping an exemplary plurality of herbal materials in water for a time period in a range of 5 minutes to 10 minutes. In an exemplary embodiment, an exemplary plurality of herbal materials may be mixed with water with a weight ratio in range of 1:2 to 1:10 (an exemplary plurality of herbal materials:water). In an exemplary embodiment, an exemplary plurality of herbal materials may be mixed with water utilizing a mixer with a stirring speed in a range of 50 rpm to 200 rpm. In an exemplary embodiment, an exemplary plurality of herbal materials may be mixed with water for a time period in a range of 5 minutes to 10 minutes. In an exemplary embodiment, an exemplary plurality of herbal materials may be dried after an exemplary washing process. In an exemplary embodiment, an exemplary plurality of herbal materials may be heated at a temperature in a range of 20° C. to 60° C. In an exemplary embodiment, an exemplary plurality of herbal materials may be heated for a time period in a range of 1 day to 5 days. In an exemplary embodiment, after drying an exemplary plurality of herbal material, an exemplary plurality of herbal materials may be grinded to form an exemplary plurality of herbal powders. In an exemplary embodiment, an exemplary plurality of herbal powders may be sieved for excluding particles larger than 200 μm. In an exemplary embodiment, an exemplary plurality of herbal powders may include particles with an average particle size in a range of 10 μm to 200 μm. In an exemplary embodiment, an exemplary herbal mixture may be produced by mixing an exemplary plurality of herbal powders in a mixer. In an exemplary embodiment, an exemplary plurality of herbal powders may be mixed in an exemplary mixer with a stirring speed in a range of 50 rpm to 200 rpm. In an exemplary embodiment, an exemplary plurality of herbal powders may be mixed in an exemplary mixer for a time period in a range of 5 minutes to 10 minutes. In an exemplary embodiment, an exemplary plurality of herbal powders may include pimpinella anisum powder, artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder. In an exemplary embodiment, each powder particle of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may have an average particle size in a range of 10 μm to 200 μm. In an exemplary embodiment, an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder may include powder of at least one of root, stem, leaf, flower, fruit, and combinations thereof of an exemplary respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa. In an exemplary embodiment, an exemplary pimpinella anisum powder may include a weight percentage in a range of 4 wt. % to 50 wt. % of a total amount of an exemplary herbal portion. In an exemplary embodiment, an exemplary pain-relief medicine may include an exemplary artemisia vulgaris powder with a weight ratio to an exemplary pimpinella anisum powder in a range of 7:50 to 15:1 (artemisia vulgaris powder:pimpinella anisum powder). In an exemplary embodiment, an exemplary pain-relief medicine may include an exemplary viola odorata powder with a weight ratio to an exemplary pimpinella anisum powder in a range of 2:25 to 25:2 (viola odorata powder:pimpinella anisum powder). In an exemplary embodiment, an exemplary pain-relief medicine may include an exemplary Cyperus species powder with a weight ratio to an exemplary pimpinella anisum powder in a range of 1:50 to 15:2 (Cyperus species powder:pimpinella anisum powder). In an exemplary embodiment, an exemplary pain-relief medicine may include an exemplary matricaria chamomilla powder with a weight ratio to an exemplary pimpinella anisum powder in a range of 1:50 to 15:4 (matricaria chamomilla powder:pimpinella anisum powder). In an exemplary embodiment, an exemplary pain-relief medicine may include an exemplary salvia officinalis powder with a weight ratio to an exemplary pimpinella anisum powder in a range of 1:50 to 25:4 (salvia officinalis powder:pimpinella anisum powder). In an exemplary embodiment, an exemplary pain-relief medicine may include an exemplary aesculus hippocastanum powder with a weight ratio to an exemplary pimpinella anisum powder in a range of 1:25 to 15:2 (aesculus hippocastanum powder:pimpinella anisum powder). In an exemplary embodiment, an exemplary pain-relief medicine may include an exemplary cymbopogon citratus powder with a weight ratio to an exemplary pimpinella anisum powder in a range of 1:25 to 5:1 (cymbopogon citratus powder:pimpinella anisum powder). In an exemplary embodiment, an exemplary pain-relief medicine may include an exemplary trachyspermum ammi powder with a weight ratio to an exemplary pimpinella anisum powder in a range of 1:50 to 5:1 (trachyspermum ammi powder:pimpinella anisum powder). In an exemplary embodiment, an exemplary pain-relief medicine may include an exemplary curcuma longa powder with a weight ratio to an exemplary pimpinella anisum powder in a range of 1:50 to 5:1 (curcuma longa powder:pimpinella anisum powder).

In further detail with respect to step 202b, step 202b of forming an herbal suspension may include mixing an exemplary herbal mixture with a hydroalcoholic solution. In an exemplary embodiment, an exemplary herbal mixture may be mixed with an exemplary hydroalcoholic solution using a mixer with a stirring speed in a range of 50 rpm to 200 rpm. In an exemplary embodiment, an exemplary herbal mixture may be mixed with an exemplary hydroalcoholic solution at a temperature in a range of 15° C. to 60° C. In an exemplary embodiment, an exemplary herbal mixture may be mixed with an exemplary hydroalcoholic solution for a time period in a range of 30 minutes to 72 hours. In an exemplary embodiment, an exemplary hydroalcoholic solution may include an alcoholic portion and water. In an exemplary embodiment, an exemplary alcoholic portion may include at least one of ethanol. In an exemplary embodiment, an exemplary hydroalcoholic solution may include an exemplary alcoholic portion with a weight ratio to water in a range of 5:95 to 99:1 (alcoholic portion:water). In an exemplary embodiment, an exemplary water may include distilled water. In an exemplary embodiment, an exemplary hydroalcoholic solution may be mixed with an exemplary herbal mixture with a weight ratio in a range of 5:1 to 100:1 (hydroalcoholic solution:herbal mixture). In an exemplary embodiment, an extract of an exemplary herbal mixture may be dissolved in an exemplary hydroalcoholic solution by mixing an exemplary hydroalcoholic solution with an exemplary herbal mixture.

In further detail with respect to step 202c, step 202c of isolating the herbal portion may include filtering an exemplary mixture of an exemplary hydroalcoholic solution and an exemplary herbal mixture. In an exemplary embodiment, an exemplary herbal mixture may be separated from an exemplary hydroalcoholic solution using a filter with pore size in a range of 1 μm to 30 μm. In an exemplary embodiment, an exemplary hydroalcoholic solution may contain dissolved extract of an exemplary herbal mixture therein. In an exemplary embodiment, an exemplary alcoholic portion of an exemplary hydroalcoholic solution may be separated from water. In an exemplary embodiment, an exemplary alcoholic portion of an exemplary hydroalcoholic solution may be separated from water using a rotary evaporator device. In an exemplary embodiment, an exemplary hydroalcoholic solution may be heated at a temperature in a range of 15° C. to 60° C. using a heater. In an exemplary embodiment, an exemplary hydroalcoholic solution may be heated for a time period in a range of 30 minutes to 72 hours. In an exemplary embodiment, water may contain an exemplary extract of an exemplary herbal mixture. In an exemplary embodiment, an exemplary extract of an exemplary herbal mixture may be dissolved in water to form an aqueous solution of an exemplary herbal extract of an exemplary herbal mixture.

In further detail with respect to step 202d, step 202d of forming a powdered-herbal extract of the herbal mixture may include evaporating water of an exemplary aqueous-herbal extract of an exemplary herbal mixture. In an exemplary embodiment, an exemplary powdered-herbal extract of an exemplary herbal mixture may be separated by freeze-drying an exemplary aqueous-herbal extract of an exemplary herbal mixture. In an exemplary embodiment, an exemplary aqueous-herbal extract may be freeze-dried at a temperature in a range of −40° C. to −60° C. In an exemplary embodiment, an exemplary aqueous-herbal extract may be freeze-dried for a time period in a range of 24 hours to 72 hours. In an exemplary embodiment, an exemplary solid-herbal extract may be powdered to from an exemplary powdered-herbal extract of an exemplary herbal mixture. In an exemplary embodiment, an exemplary powdered-herbal extract of an exemplary herbal mixture may include powder particles with an average particle size in a range of 10 μm to 200 μm. In an exemplary embodiment, an exemplary powdered-herbal extract of an exemplary herbal mixture may include powdered-herbal extract of pimpinella anisum powder, artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder. In an exemplary embodiment, exemplary powder particles of an exemplary powdered-herbal extract of an exemplary herbal mixture may include powder particles of each of an exemplary pimpinella anisum powder, an exemplary artemisia vulgaris powder, an exemplary viola odorata powder, an exemplary Cyperus species powder, an exemplary matricaria chamomilla powder, an exemplary salvia officinalis powder, an exemplary aesculus hippocastanum powder, an exemplary cymbopogon citratus powder, an exemplary trachyspermum ammi powder, and an exemplary curcuma longa powder with an average particle size in a range of 10 μm to 200 μm. In an exemplary embodiment, an exemplary powdered-herbal extract of an exemplary herbal mixture may be used as an exemplary herbal portion for producing an exemplary pain-relief medicine. In an exemplary embodiment, an exemplary powdered-herbal extract of an exemplary herbal mixture may be used to form an exemplary pain-relief medicine in a form of tablet, capsule, tea bag, suppository, effervescent tablet, and combinations thereof.

Referring back to FIG. 2A, in further detail with respect to step 204, step 204 of forming a supplementary portion may include forming a plurality of supplementary powders of a plurality of supplementary materials. In an exemplary embodiment, an exemplary plurality of supplementary powders may be produced by grinding a plurality of supplementary materials to an average particle size in a range of 1 μm to 200 μm. In an exemplary embodiment, an exemplary supplementary powders may be mixed in a mixer with a stirring speed in a range of 50 rpm to 200 rpm. In an exemplary embodiment, an exemplary plurality of supplementary powders may be mixed in a mixer for a time period in a range of 5 minutes to 10 minutes. In an exemplary embodiment, an exemplary plurality of supplementary powders may include coenzyme Q₁₀ powder, carnitine powder, magnesium powder, α-lipoic acid powder, zinc powder, iron powder, vitamin C powder, vitamin E powder, vitamin D powder, vitamin B powder, and caffeine powder. In an exemplary embodiment, an exemplary supplementary powders may include particles with an average particle size in a range of 1 μm to 200 μm. In an exemplary embodiment, an exemplary vitamin B may include at least one of B1 (thiamin), B2 (riboflavin), B3 (niacin), B5 (pantothenic acid), B6 (pyridoxine), B7 (biotin), B9 (folate [folic acid]), B12 (cobalamin), and combinations thereof.

In further detail with respect to step 206, step 206 of forming a tablet mixture may include mixing an exemplary herbal portion with an exemplary supplementary portion. In an exemplary embodiment, forming an exemplary tablet mixture may include mixing an exemplary powdered-herbal extract of an exemplary herbal mixture with an exemplary supplementary portion. In an exemplary embodiment, an exemplary powdered-herbal extract of an exemplary herbal mixture may be mixed with an exemplary supplementary portion using a mixer with a stirring speed in a range of 50 rpm to 200 rpm. In an exemplary embodiment, an exemplary powdered-herbal extract of an exemplary herbal mixture may be mixed with an exemplary supplementary portion for a time period in a range of 5 minutes to 10 minutes. In an exemplary embodiment, at least one adhesive substance may be added into an exemplary mixture of an exemplary powdered-herbal extract of an exemplary herbal mixture and an exemplary supplementary portion. In an exemplary embodiment, an exemplary adhesive substance may form connections among exemplary particles of an exemplary tablet mixture. In an exemplary embodiment, an exemplary herbal portion, an exemplary supplementary portion, and an exemplary at least one adhesive substance may be mixed together in a mixer with a stirring speed in a range of 50 rpm to 200 rpm. In an exemplary embodiment, an exemplary herbal portion, an exemplary supplementary portion, and an exemplary at least one adhesive substance may be mixed together in a mixer for a time period in a range of 5 minutes to 10 minutes. In an exemplary embodiment, an exemplary at least one adhesive substance may include at least one of starch, PVP, and combinations thereof. In an exemplary embodiment, an exemplary pain-relief medicine may include an exemplary at least one adhesive substance with a weight percentage of a total amount of an exemplary pain-relief medicine in a range of 2 wt. % to 10 wt. %.

In further detail with respect to step 208, step 208 of applying a pressure on an exemplary tablet mixture may include applying a pressure on an exemplary tablet mixture utilizing a press device. In an exemplary embodiment, an exemplary pressure applied on an exemplary tablet mixture may be in a range of 2500 kPa to 6500 kPa. In an exemplary embodiment, an exemplary tablet mixture may be pressed for a time period in a range of 1 second to 10 seconds. In an exemplary embodiment, a predetermined amount of an exemplary tablet mixture may be used to form an exemplary tablet of an exemplary pain-relief medicine. In an exemplary embodiment, an exemplary predetermined amount of an exemplary tablet mixture may be in a range of 4 gr to 8 gr. In an exemplary embodiment, an exemplary tablet mixture may be pressed at a temperature in a range of 20° C. to 25° C. In an exemplary embodiment, an exemplary pain-relief medicine may be taken with a weight in a range 4 gr to 8 gr. In an exemplary embodiment, an exemplary pain-relief medicine may be taken for 1 day to 3 days. In an exemplary embedment, an exemplary pain-relief medicine may be used for relieving at least one of headache, sleep disorder, menstrual problems, digestive problems, mental problems, hormonal problems, increased brain pressure, and combinations thereof.

### EXAMPLE 1

### Producing a Pain-Relief Medicine for Relieving Headache

For producing a pain-relief medicine in a form of tablet, a method similar to method 200 and/or method 210 was used. In the first step, a plurality of herbal powders was prepared. For preparing an exemplary plurality of herbal powders seeds of Pimpinella anisum, arial parts of Artemisia vulgaris, flowers of Viola odorata, leaves of Cyperus species, flowers of Matricaria chamomilla, leaves or flowers of Salvia officinalis, fruits of Aesculus hippocastanum, leaves of Cymbopogon citratus, seeds of Trachyspermum ammi, and roots of Curcuma longa were dried at a temperature of 20° C. to 60° C. for 1 day to 5 days. An exemplary pimpinella anisum, an exemplary artemisia vulgaris, an exemplary viola odorata, an exemplary Cyperus species, an exemplary matricaria chamomilla, an exemplary salvia officinalis, an exemplary aesculus hippocastanum, an exemplary cymbopogon citratus, an exemplary trachyspermum ammi, and an exemplary curcuma longa were grinded. Exemplary herbal powders had average particle size in a range of 100 μm to 600 μm. An exemplary pain-relief medicine may include pimpinella anisum powder with a weight percentage of a total amount of an exemplary pain-relief medicine of 5 wt. % to 15 wt. %. An exemplary pain-relief medicine may include artemisia vulgaris powder and the pimpinella anisum powder with a weight ratio of 1:5 (artemisia vulgaris:pimpinella anisum). An exemplary pain-relief medicine may include viola odorata powder and an exemplary pimpinella anisum powder with a weight ratio of 2:5 (viola odorata:pimpinella anisum). An exemplary pain-relief medicine may include the Cyperus species powder and an exemplary pimpinella anisum with a weight ratio of 1:1 (Cyperus species:pimpinella anisum). An exemplary pain-relief medicine may include matricaria chamomill powder and an exemplary pimpinella anisum with a weight ratio of 1:1 (matricaria chamomill:pimpinella anisum). An exemplary pain-relief medicine may include salvia officinalis powder and an exemplary pimpinella anisum with a weight ratio of 1:1 (salvia officinalis:pimpinella anisum). An exemplary pain-relief medicine may include aesculus hippocastanum powder and an exemplary pimpinella anisum powder with a weight ratio of 1:50 (aesculus hippocastanum:pimpinella anisum). An exemplary pain-relief medicine may include cymbopogon citratus powder and pimpinella anisum powder with a weight ratio of 1:25 (cymbopogon citratus:pimpinella anisum). An exemplary pain-relief medicine may include trachyspermum ammi powder and pimpinella anisum powder with a weight ratio of 1:50 (trachyspermum ammi:pimpinella anisum). An exemplary pain-relief medicine may include curcuma longa powder and an exemplary pimpinella anisum powder with a weight ratio of 1:50 (curcuma long:pimpinella anisum).

Exemplary herbal powders were mixed with a hydroalcoholic solution with a weight ratio of 1:4 (herbal powder:hydroalcoholic solution). The mixture of exemplary herbal powders and an exemplary hydroalcoholic solution was stirred with a stirring speed of 120 rpm for 48 hours. An extract of an exemplary plurality of herbal powders was separated utilizing a rotary evaporator device. An exemplary mixture of an exemplary herbal powders and an exemplary hydroalcoholic solution was heated at a temperature of 35° C. for 240 minutes in an exemplary rotary evaporator device. An aqueous phase of an exemplary hydroalcoholic solution was freeze-dried at a temperature of −40° C. for 48 hours to form a powdered-herbal extract of an exemplary herbal mixture.

A supplementary portion was mixed with an exemplary herbal portion with a weight ratio of 1:1 (herbal portion:supplementary portion). An exemplary supplementary portion may include coenzyme Q₁₀ powder, carnitine powder, magnesium powder, α-lipoic acid powder, zinc powder, iron powder, vitamin C powder, vitamin E powder, vitamin D powder, vitamin B powder, and caffeine powder. An exemplary pain-relief medicine may include coenzyme Q₁₀ powder and an exemplary pimpinella anisum powder with a weight ratio of 1:1 (coenzyme Q₁₀:pimpinella anisum). An exemplary pain-relief medicine may include carnitine powder and an exemplary pimpinella anisum powder with a weight ratio of 1:1 (carnitine powder:pimpinella anisum). An exemplary pain-relief medicine may include magnesium powder and an exemplary pimpinella anisum powder with a weight ratio of 1:1 (magnesium powder:pimpinella anisum). An exemplary pain-relief medicine may include α-lipoic acid powder and an exemplary pimpinella anisum powder with a weight ratio of 1:1 (α-lipoic acid powder:pimpinella anisum). An exemplary pain-relief medicine may include zinc powder and an exemplary pimpinella anisum powder with a weight ratio of 1:5 (zinc powder:pimpinella anisum). An exemplary pain-relief medicine may include iron powder and an exemplary pimpinella anisum powder with a weight ratio of 1:5 (iron powder:pimpinella anisum). An exemplary pain-relief medicine may include vitamin C powder and an exemplary pimpinella anisum powder with a weight ratio of 1:1 (vitamin C powder:pimpinella anisum). An exemplary pain-relief medicine may include vitamin E powder and an exemplary pimpinella anisum powder with a weight ratio of 1:10 (vitamin E powder:pimpinella anisum). An exemplary pain-relief medicine may include vitamin D powder and an exemplary pimpinella anisum powder with a weight ratio of 0.00125:50 (vitamin D powder:pimpinella anisum). An exemplary pain-relief medicine may include vitamin B powder and an exemplary pimpinella anisum powder with a weight ratio of 1:10 (vitamin B powder:pimpinella anisum). An exemplary pain-relief medicine may include caffeine powder and an exemplary pimpinella anisum with a weight ratio of 1:5 (caffeine powder:pimpinella anisum). For the final step, 5 gr starch as an adhesive substance was added to an exemplary mixture of an exemplary herbal portion and an exemplary supplementary portion to form a tablet mixture. An exemplary adhesive substance may form adhesive bonds among particles of an exemplary mixture of an exemplary herbal portion and an exemplary supplementary portion. After forming an exemplary tablet mixture 0.4 gr to 0.8 gr of an exemplary tablet mixture was pressed using a press device with a pressure of 2500 kPa.

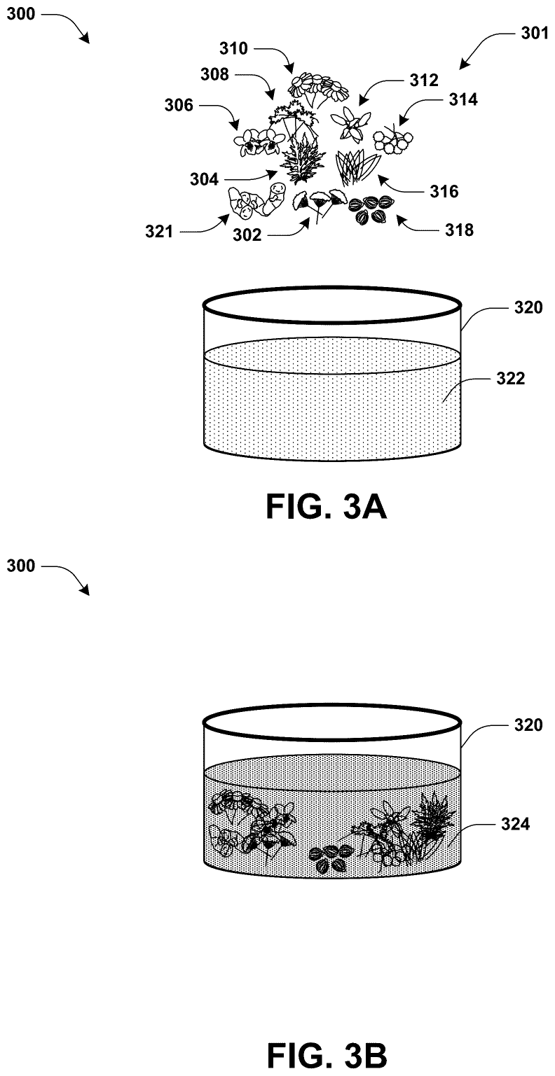

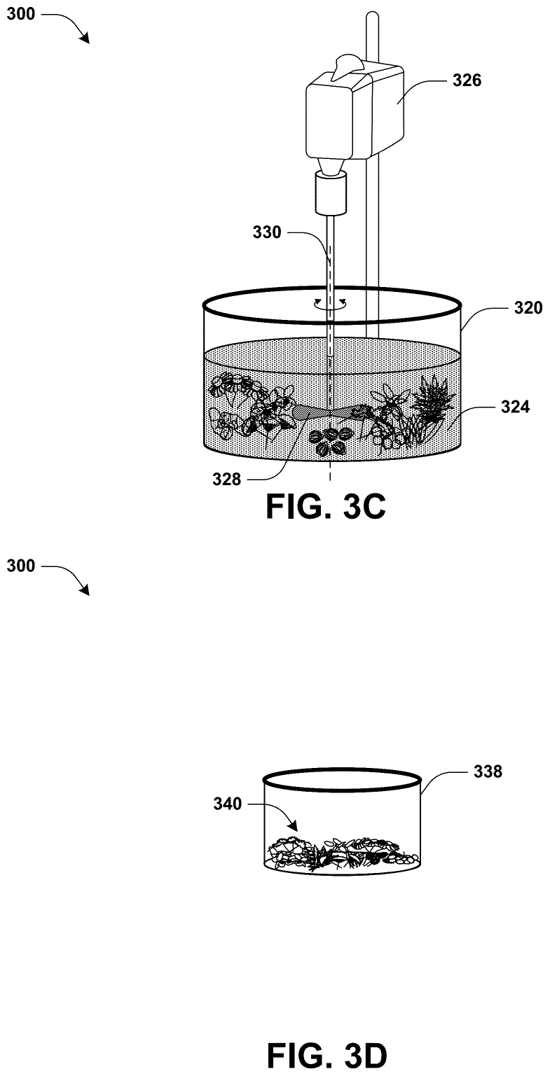

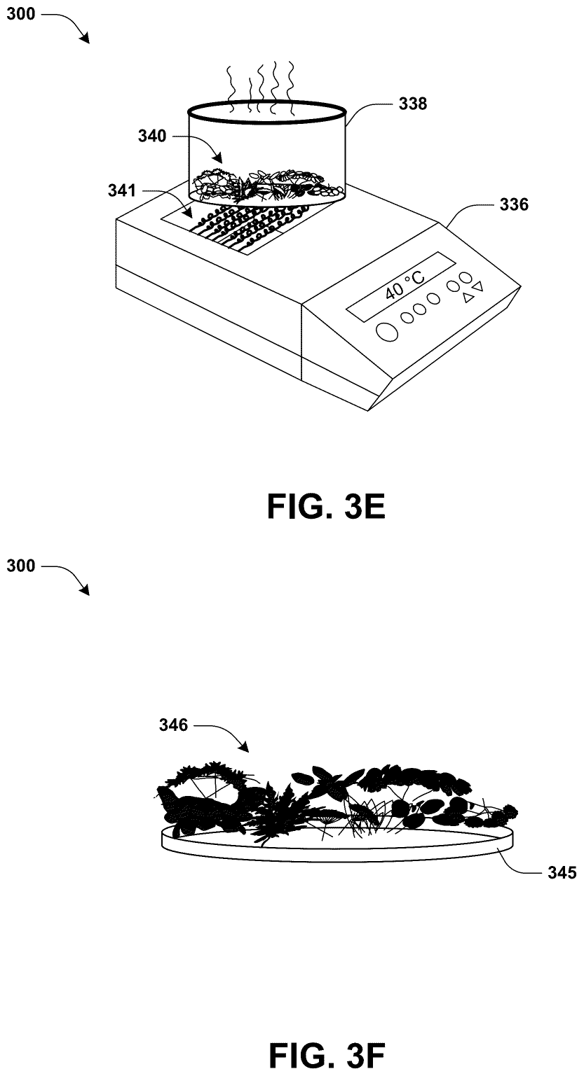

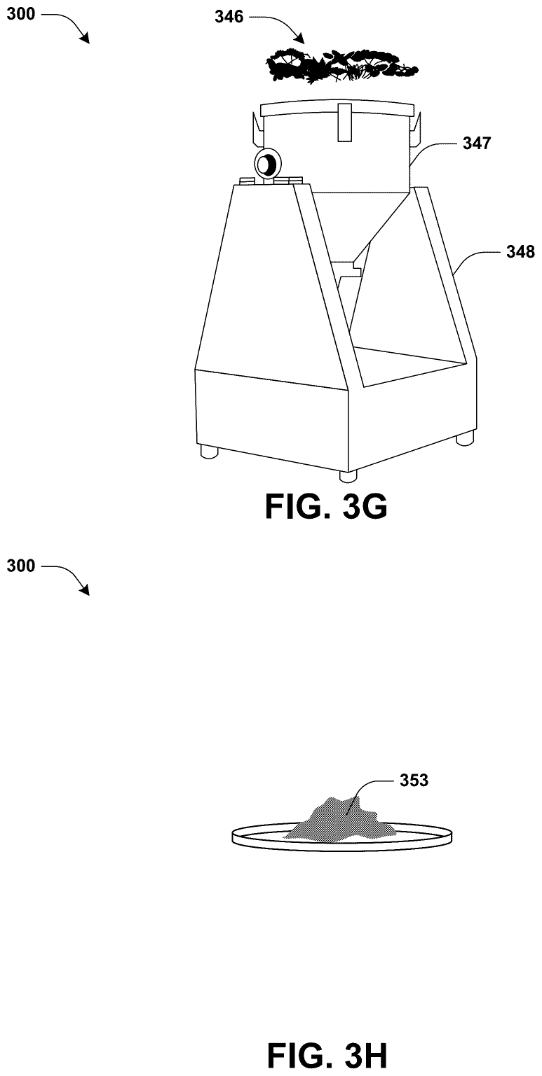

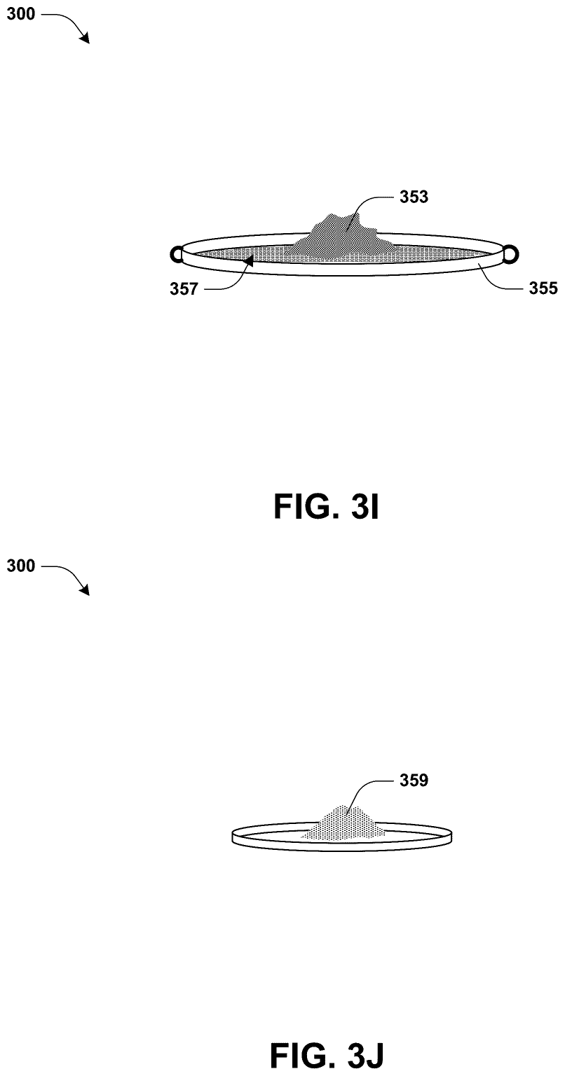

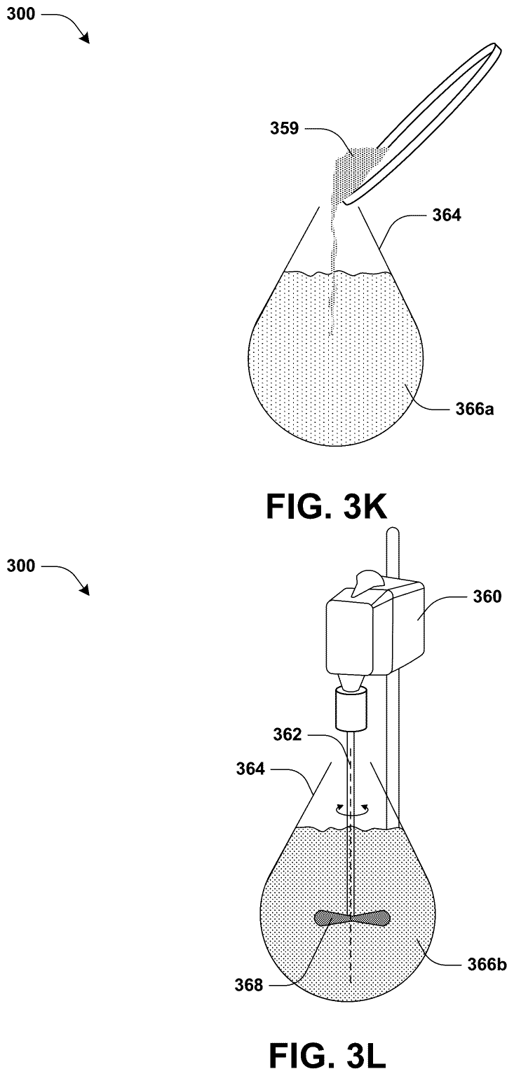

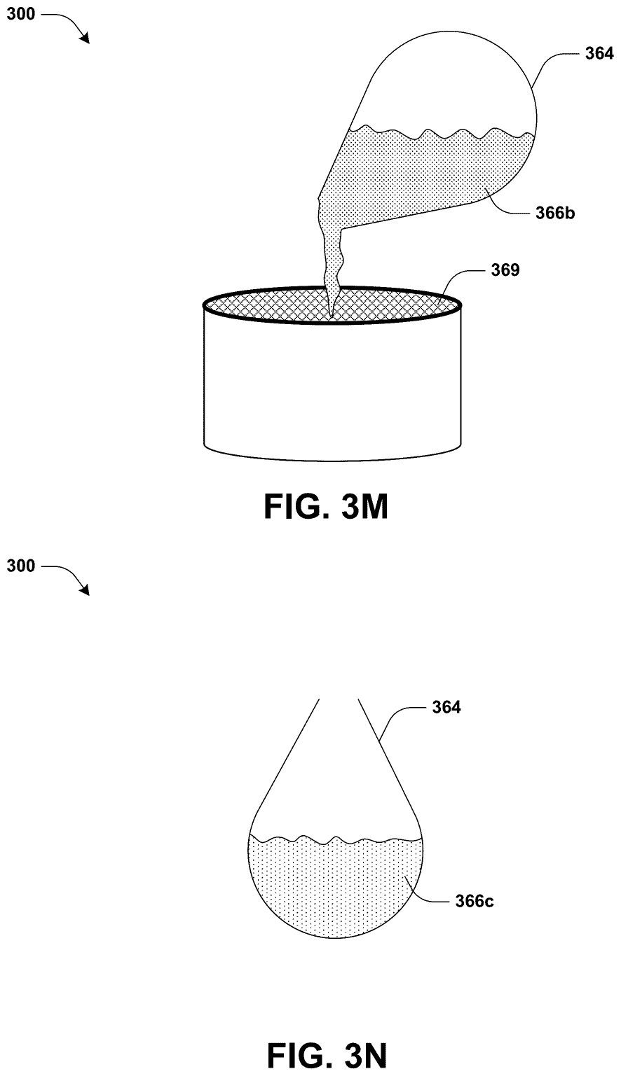

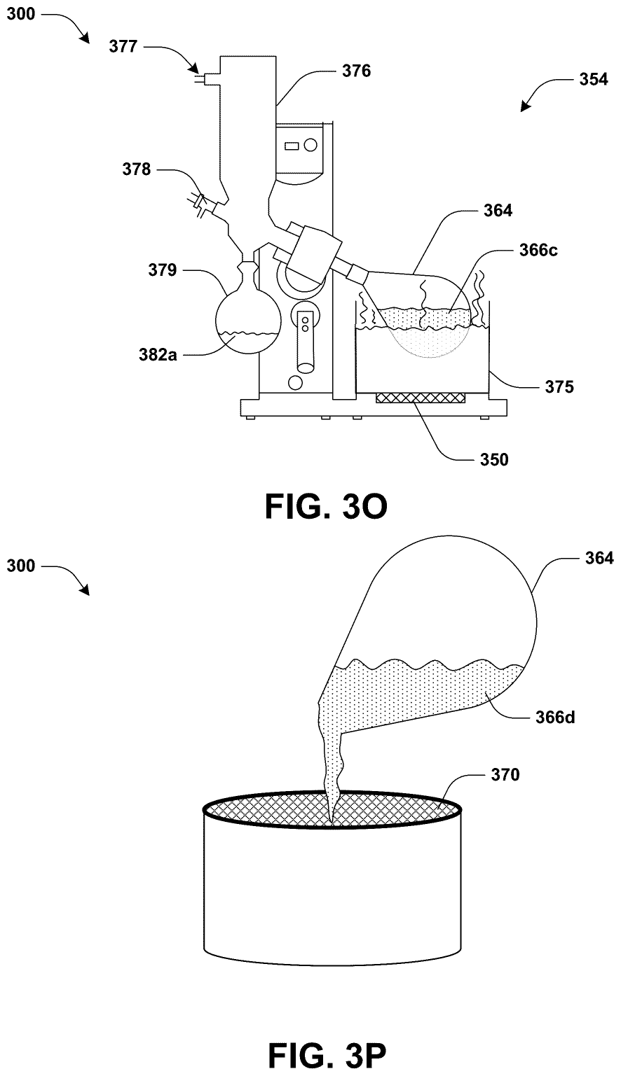

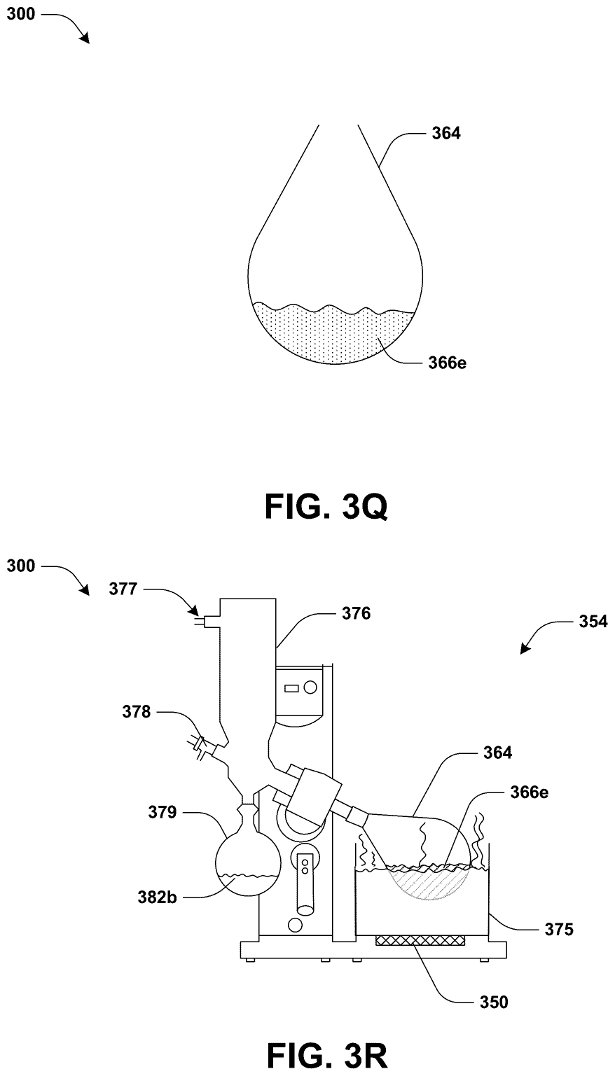

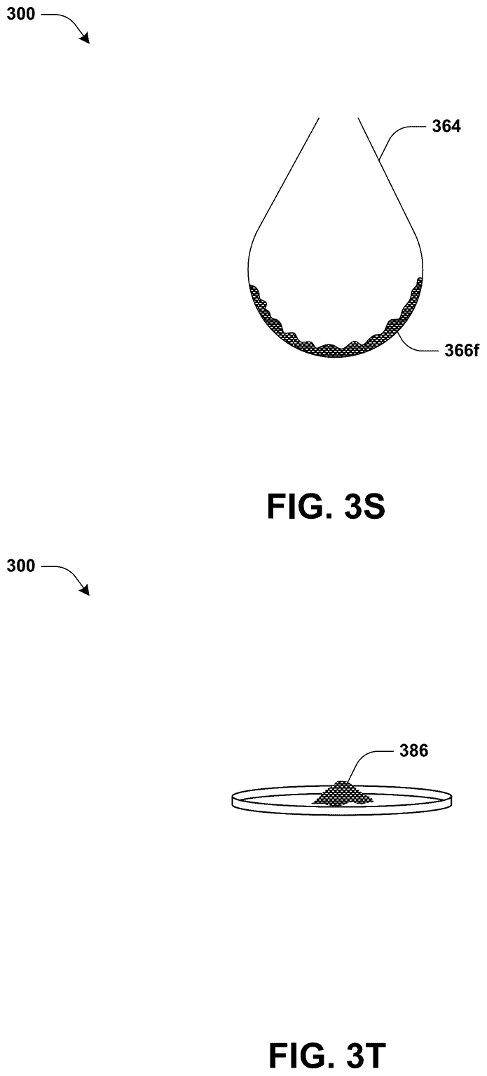

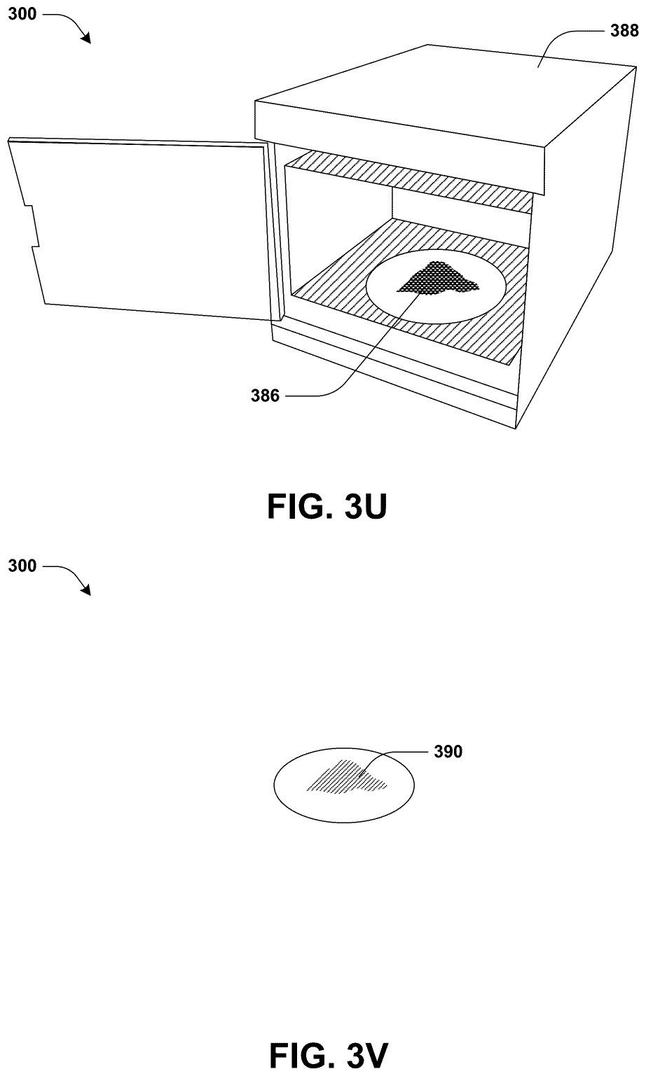

FIGS. 3A-3V illustrate an example scenario 300 in which an herbal portion is produced. In FIGS. 3A-3B, one or more herbal compounds 301 may be placed inside a container 320 (e.g., a dish, a bucket, etc.). The container 320 may comprise a liquid 322 (e.g. water, distilled water, etc.). In some examples, the one or more herbal compounds comprise a pimpinella anisum 302, an artemisia vulgaris 304, a viola odorata 306, a Cyperus species 308, a matricaria chamomilla 310, a salvia officinalis 312, an aesculus hippocastanum 314, a cymbopogon citratus 316, a trachyspermum ammi 318 and/or a curcuma longa 321. In some examples, the one or more herbal compounds 301 may be dipped in the liquid 322 for a time period (e.g., in a range of about 2 minutes to about 20 minutes) to produce an herbal mixture 324 associated with the one or more herbal compounds 301. In some examples, the one or more herbal compounds 301 may be dipped in the liquid 322 for a time period (e.g., in a range of about 5 minutes to about 10 minutes) to produce the herbal mixture 324 associated with the one or more herbal compounds 301. As shown in FIG. 3C, a stirring device 326 (e.g., an overhead stirring device) may be utilized to stir the herbal mixture for a time period (e.g., in a range of about 5 minutes to 10 minutes) with the stirring speed of about 50 rpm to about 200 rpm. In some examples, the stirring device 326 comprises a rotary unit 328 (e.g., a propeller, a magnetic propeller, etc.) to rotate with the stirring speed around a rotational axis 330. In some examples, the one or more herbal compounds 301 turn into one or more mixed herbal compounds (upon being mixed by the stirring device 326, for example).

In FIG. 3D, the one or more mixed herbal compounds (shown with reference number 340) are placed inside a container 338. In some examples, the herbal mixture 324 may be filtered (e.g., sieved) to separate the one or more mixed herbal compounds 340 from the liquid 322. In FIG. 3E, the one or more mixed herbal compounds 340 may be heated utilizing a heater 336. In some examples, the heater 336 comprises one or more heating elements 341 configured to apply heat which may dry the one or more mixed herbal compounds 340. In some examples, the one or more mixed herbal compounds 340 may be heated, by the one or more heating elements 341, by configuring the heater 336 to heat at a temperature in a range of about 10° C. to about 100° C. for a time period in a range of about an hour to about 10 days. In some examples, the one or more mixed herbal compounds 340 may be heated, by the one or more heating elements 341, by configuring the heater 336 to heat at a temperature in a range of about 20° C. to about 60° C. for a time period in a range of about 1 day to about 5 days. In some examples, after receiving heat from the one or more heating elements 341, the one or more mixed herbal compounds 340 may turn into one or more dried compounds 346.

In FIG. 3F, the one or more dried herbal compounds 346 are placed inside a container 345. As shown in FIG. 3G, the one or more dried herbal compounds 346 may be placed inside a grinder 348 (e.g., a mixer device). The grinder 348 may comprise a grinding unit 347 to grind the one or more dried herbal compounds 346. In some examples, the grinder 348 produces an herbal powder 353 (shown in FIG. 3H). In some examples, the herbal powder 353 may comprise herbal particles with an average size in a range of about 10 μm to about 500 μm.

In FIG. 3I, the herbal powder 353 are placed inside a filter 355 (e.g., a sieve). In some examples, the filter 355 comprises a plurality of holes 357 (e.g., a plurality of circular holes). In some examples, a diameter of the plurality of holes 357 is about 200 μm. In some examples, the plurality of holes 357 are used to exclude particles of the herbal powder 353 with a size larger than about 200 μm and/or to provide a filtered herbal powder 359 (as shown in FIG. 3J). In some examples, the filtered herbal powder 359 may comprise herbal particles with an average size in a range of about 10 μm to about 200 μm.

In FIG. 3K, the filtered herbal powder 359 may be added to (e.g., poured into) a hydroalcoholic solution 366a inside a container 364 (e.g., a dish, a beaker, a laboratory glass, etc.). In some examples, a ratio of the herbal powder 353 to the hydroalcoholic solution 366a by weight is in a range of about 1:3 to about 1:5. In some examples, the hydroalcoholic solution 366a may comprise an alcoholic portion and/or a water portion. In some examples, the alcoholic portion comprises ethanol. In some examples, the water portion comprises distilled water. In some examples, a ratio of the alcoholic portion to the water portion by weight is in a range of about 5:95 to about 99:1. In FIG. 3L, the hydroalcoholic solution 366a may be poured into a stirring device 360 and/or stirred by the stirring device 360 for a time period of, for example, about 6 hours to about 72 hours. In some examples, the hydroalcoholic solution 366a may be poured into the stirring device 360 and/or stirred by the stirring device 360 for a time period of, for example, about 12 hours to about 48 hours. In some examples, the stirring device 360 (e.g., an overhead stirring device) may be utilized to stir the hydroalcoholic solution 366a and/or the filtered herbal powder 359 for a time period in a range of, for example, about 5 hours to about 72 hours with the stirring speed of, for example, about 30 rpm to about 300 rpm. In some examples, the stirring device 360 (e.g., an overhead stirring device) may be utilized to stir the hydroalcoholic solution 366a and/or the filtered herbal powder 359 for a time period in a range of, for example, about 12 hours to about 48 hours with the stirring speed of, for example, about 50 rpm to about 200 rpm. In some examples, the stirring device 360 comprises a rotary unit 368 (e.g., a propeller, a magnetic propeller, etc.) to rotate with the stirring speed around a rotational axis 362. In some examples, the hydroalcoholic solution 366a may be added to the filtered herbal powder 359 and/or stirred by the stirring device 360 to produce an herbal suspension 366b. In FIG. 3M, the herbal suspension 366b may be filtered utilizing a filter 369 (e.g., a Whatman filter paper). In some examples, the filter 369 may comprise a pore size (e.g., diameter of pores, diameter of holes, diameter of circular holes, etc.) within a range of, for example, about 1 μm to about 30 μm. In an example, the herbal suspension 366b may be filtered one or more times (e.g., three times). In some examples, the process of filtering the herbal suspension 366b produces a filtered herbal suspension 366c (shown in FIG. 3N).

FIG. 3O illustrates a rotary evaporation device 354, according to some embodiments. In some examples, the filtered herbal suspension 366c may be evaporated utilizing the rotary evaporation device 354. In some examples, the rotary evaporation device 354 may be utilized to remove a portion of the water portion of the filtered herbal suspension 366c, and/or a portion of the alcoholic portion of the filtered herbal suspension 366c. In some examples, the rotary evaporation device 354 may comprise a heater 350 configured to heat the filtered herbal suspension 366c. In some examples, the heater 350 may be configured to heat the filtered herbal suspension 366c at a temperature in a range of, for example, about 10° C. to about 80° C. for a time period in a range of, for example, about 15 minutes to about 2 hours. In some examples, the heater 350 may be configured to heat the filtered herbal suspension 366c at a temperature in a range of, for example, about 20° C. to about 60° C. (e.g., 35° C.) for a time period in a range of, for example, about 30 minutes to about 1 hour. In some examples, the rotary evaporation device 354 may comprise a heating bath 375. In some examples, the heating bath 375 comprises a liquid (e.g., water, distilled water, etc.). In some examples, the container 364 and/or the filtered herbal suspension 366c inside the container 364 may be soaked inside the liquid. In some examples, the rotary evaporation device 354 generates a vacuum via using a vacuum pump 378. In some examples, the rotary evaporation device 354 may be utilized to rotate the container 364 and/or the filtered herbal suspension 366c inside the container 364 for a time period in a range of, for example, about 15 minutes to about 3 hours with a rotational speed of, for example, about 10 rpm to about 400 rpm. In some examples, the rotary evaporation device 354 may be utilized to rotate the container 364 and/or the filtered herbal suspension 366c inside the container 364 for a time period in a range of, for example, about 30 minutes to about 1 hour with a rotational speed of, for example, about 30 rpm to about 300 rpm. In some examples, the heater 350 may heat the liquid inside the heating bath 375. In some examples, the heating bath 375 may heat the container 364 and/or the filtered herbal suspension 366c inside the container 364. In some examples, the heater 350 and/or the heating bath 375 may cause a portion of the water portion of the filtered herbal suspension 366c, and/or a portion of the alcoholic portion of the filtered herbal suspension 366c to evaporate. In some examples, a portion of the water portion of the filtered herbal suspension 366c turns into a water vapor (upon being heated by the heater 350 and/or the heating bath 375, for example). In some examples, a portion of the alcoholic portion of the filtered herbal suspension 366c turns into an alcohol vapor (upon being heated by the heater 350 and/or the heating bath 375, for example). In some examples, the water vapor and/or the alcohol vapor enter a condenser 376 (e.g., a condensing tower). In some examples, the water vapor may be cooled down (e.g., turned into a distilled water 382a) and collected by a receiving flask 379. In some examples, the alcohol vapor may exit the rotary evaporation device 354 via an exhaust 377 (e.g., an outlet). In some examples, the filtered herbal suspension 366c turns into an aqueous-herbal extract 366d (upon being processed by the rotary evaporation device 354, for example).

In some examples, an aqueous-herbal extract may be produced via using one or more techniques. For example, the herbal power 353 may be added to a portion of water to produce an herbal powder solution. In some examples, a ratio of the herbal powder 353 to the portion of the water by weight is in a range of about 1:3 to about 1:5. In some examples, the herbal powder solution may be poured in a stirring device (e.g., the stirring device 360) and/or stirred by the stirring device for a time period of about 6 hours to about 72 hours. In some examples, the herbal powder solution may be poured in the stirring device and/or stirred by the stirring device for a time period of about 12 hours to about 48 hours. In some examples, the herbal powder solution may be filtered utilizing a filter (e.g., the filter 369). In some examples, the filter may comprise pore size (e.g., diameter of pores, diameter of holes, diameter of circular holes, etc.) within a range of about 1 μm to about 30 μm. In an example, the herbal powder solution may be filtered one or more times (e.g., three times).

In FIG. 3P, the aqueous-herbal extract 366d may be filtered utilizing a filter 370 (e.g., a Whatman filter paper). In some examples, the filter 370 may comprise pore size (e.g., diameter of pores, diameter of holes, diameter of circular holes, etc.) within a range of, for example, about 1 μm to about 30 μm. In an example, the aqueous-herbal extract 366d may be filtered one or more times (e.g., two times, three times, four times, etc.). In some examples, the process of filtering the aqueous-herbal extract 366d (utilizing the filter 370, for example) produces a filtered aqueous-herbal extract 366e (shown in FIG. 3Q).

FIG. 3R illustrates the rotary evaporation device 354, according to some embodiments. In some examples, the filtered aqueous-herbal extract 366e may be evaporated utilizing the rotary evaporation device 354. In some examples, the rotary evaporation device 354 may be utilized to remove a portion of a water portion (e.g., some of the water portion, or an entirety of the water portion) of the filtered aqueous-herbal extract 366e, and/or a portion of an alcoholic portion (e.g., some of the alcoholic portion, or an entirety of the alcoholic portion) of the filtered aqueous-herbal extract 366e. In some examples, the rotary evaporation device 354 may comprise the heater 350 to heat the filtered aqueous-herbal extract 366e. In some examples, the heater 350 may be configured to heat the filtered aqueous-herbal extract 366e at a temperature in a range of, for example, about 10° C. to about 80° C. for a time period in a range of, for example, about 1 hour to about 10 hours. In some examples, the heater 350 may be configured to heat the filtered aqueous-herbal extract 366e may be heated at a temperature in a range of, for example, about 20° C. to about 60° C. (e.g., 35° C.) for a time period in a range of, for example, about 2 hours to about 8 hours. In some examples, the rotary evaporation device 354 may comprise the heating bath 375. In some examples, the heating bath 375 comprises a liquid (e.g., water, distilled water, etc.). In some examples, the container 364 and/or the filtered aqueous-herbal extract 366e inside the container 364 may be soaked inside the liquid. In some examples, the rotary evaporation device 354 generates a vacuum via using the vacuum pump 378. In some examples, the rotary evaporation device 354 may be utilized to rotate the container 364 and/or the filtered aqueous-herbal extract 366e inside the container 364 for a time period in a range of, for example, about 1 hour to about 10 hours with a rotational speed of, for example, about 10 rpm to about 400 rpm. In some examples, the rotary evaporation device 354 may be utilized to rotate the container 364 and/or the filtered aqueous-herbal extract 366e inside the container 364 for a time period in a range of, for example, about 2 hours to about 8 hours with a rotational speed of, for example, about 30 rpm to about 300 rpm. In some examples, the heater 350 may heat the liquid inside the heating bath 375. In some examples, the heating bath 375 may heat the container 364 and/or the filtered aqueous-herbal extract 366e inside the container 364. In some examples, the heater 350 and/or the heating bath 375 may cause a portion of a water portion (e.g., some of the water portion, or an entirety of the water portion) of the filtered aqueous-herbal extract 366e, and/or a portion of an alcoholic portion of the filtered aqueous-herbal extract 366e to evaporate. In some examples, a portion of the water portion of the filtered aqueous-herbal extract 366e turns into a water vapor (upon being heated by the heater 350 and/or the heating bath 375, for example). In some examples, a portion of the alcoholic portion (e.g., some of the alcoholic portion, or an entirety of the alcoholic portion) of the filtered aqueous-herbal extract 366e turns into an alcohol vapor (upon being heated by the heater 350 and/or the heating bath 375, for example). In some examples, the water vapor and/or the alcohol vapor enter the condenser 376 (e.g., a condensing tower). In some examples, the water vapor may be cooled down (e.g., turned into a distilled water 382b) and/or collected by the receiving flask 379. In some examples, the alcohol vapor may exit the rotary evaporation device 354 via the exhaust 377 (e.g., an outlet). In some examples, the exiting of the alcohol vapor may cause the filtered aqueous-herbal extract 366e to turn into a solid-herbal extract 366f (shown in FIG. 3S), for example based upon being evaporated by the rotary evaporation device 354. In some examples, the solid-herbal extract 366f may be attached to a portion of inside wall of the container 364 in a form of a sediment. In some examples, the solid-herbal extract 366f may be gathered from the container 364. In some examples, a mixer device may be utilized to turn the solid-herbal extract 366f into the powdered-herbal extract 386 (shown in FIG. 3T). In some examples, the powdered-herbal extract 386 comprises particles with an average size in a range of about 10 μm to about 500 μm.

In FIG. 3U, a freeze dryer device 388 may be utilized to freeze dry the powdered-herbal extract 386. In some examples, the powdered-herbal extract 386 is freeze dried at a temperature in a range of, for example, about −10° C. to about −100° C. for a time period in a range of, for example, about 2 hours to about 12 hours. In an example, the powdered-herbal extract 386 is freeze dried at a temperature in a range of, for example, about −20° C. to about −90° C. for a time period in a range of, for example, about 3 hours to about 10 hours. In some examples, the powdered-herbal extract 386 is freeze dried at a temperature in a range of, for example, about −20° C. to about −70° C. for a time period in a range of, for example, about 10 hours to about 72 hours. In an example, the powdered-herbal extract 386 is freeze dried at a temperature in a range of, for example, about −30° C. to about −60° C. for a time period in a range of, for example, about 24 hours to about 56 hours. In an example, the powdered-herbal extract 386 may be freeze dried one or more times (e.g., two times). In some examples, the powdered-herbal extract 386 may be utilized as the herbal portion 390 (shown in FIG. 3V) to produce a pain-relief medicine. In some examples, the herbal portion 390 comprises particles with an average size in a range of, for example, about 10 μm to about 200 μm.

In some examples, an aqueous-herbal extract (e.g., the aqueous-herbal extract 366d) may be sprayed (e.g., atomized) inside a spray dryer device with a volumetric flow rate in a range of, for example, about 2 milliliters per minute to about 10 milliliters per minute. In some examples, the spray dryer device may comprise a drying gas. In some examples, the drying gas may enter the spray dryer device with a temperature in a range of, for example, about 120° C. to about 180° C. and/or may exit the spray dryer device with a temperature in a range of, for example, about 80° C. to about 140° C. In some examples, the drying gas may heat the sprayed aqueous-herbal extract and/or remove water portion of the aqueous-herbal extract. In an example, after removing the water portion of the aqueous-herbal extract, a solid-herbal extract may remain inside a tank of the spray dryer device. In some examples, a mixer device may be utilized to turn the solid-herbal extract into a powdered-herbal extract. In some examples, the powdered-herbal extract may be utilized as an herbal portion to produce a pain-relief medicine. In some examples, the herbal portion comprises particles with an average size in a range of, for example, about 10 μm to about 200 μm.

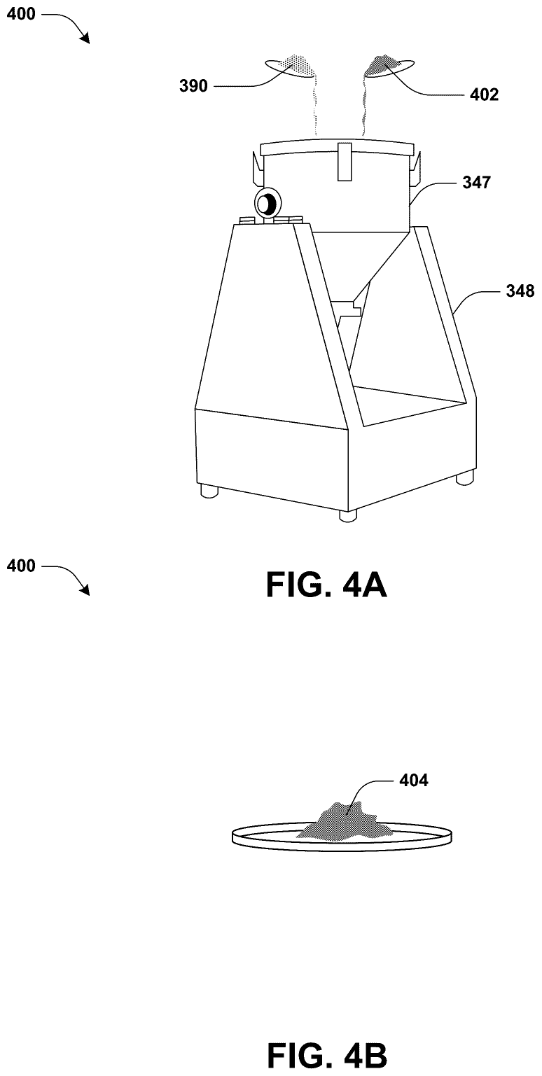

FIG. 4A-4B illustrate an example scenario 400 in which a pain-relief medicine is produced. In FIG. 4A, the grinder 348 may be utilized to produce a pain-relief medicine 404 (shown in FIG. 4B). in an example, the mixer device comprises the grinding unit 347 to combine (e.g., grind, mix, etc.) the herbal portion 390 with a supplementary portion 402. In some examples, the herbal portion 390 may comprise a pimpinella anisum powder, an artemisia vulgaris powder, a viola odorata powder, a Cyperus species powder, a matricaria chamomilla powder, a salvia officinalis powder, an aesculus hippocastanum powder, a cymbopogon citratus powder, a trachyspermum ammi powder and/or a curcuma longa powder. In some examples, the herbal portion 390 comprises particles with an average particle size in a range of, for example, about 10 μm to about 200 μm. In some examples, the supplementary portion 402 may comprise coenzyme Q₁₀ (e.g., a cofactor) powder, carnitine powder, α-lipoic acid powder, zinc powder, iron powder, vitamin C powder, vitamin E powder, vitamin D powder, vitamin B powder and/or caffeine powder. In some examples, the supplementary portion 402 comprises particles with an average particle size in a range of, for example, about 10 μm to about 200 μm. In some examples, a ratio of the herbal portion 390 to the supplementary portion 402 by weight is between about 1:7 to about 7:1. In an example, a ratio of the herbal portion 390 to the supplementary portion 402 by weight is between about 3:5 to about 5:3. In FIG. 4B based upon mixing the herbal portion 390 and 402, the pain-relief medicine 404 is produced.

In some examples, the pain-relief medicine may be utilized to produce a capsule, a syrup, a tea bag, a suppository and/or an effervescent tablet. In some examples, the pain-relief medicine may comprise the herbal portion 390 and the supplementary portion 402 by weight ratio about 1:1. For example, based upon utilizing a capsule filling machine (e.g., manual capsule filler, automatic capsule filling machine), one or more capsules associated with the pain-relief medicine are provided. In some examples, a weight of a capsule of the one or more capsules may be in a range of, for example, about 250 milligrams (mg) to about 1000 mg. In some examples, the one or more capsules may be preserved in a temperature lower than about 30° C. In some examples, the capsule may be given to a patient, one or more times (e.g., six times) a day.

### INDUSTRIAL APPLICABILITY

The present disclosure may include a pain-relief medicine for relieving headache. An exemplary pain-relief medicine is free of chemical compositions with harmful side effects. An exemplary pain-relief medicine may include an herbal portion and a supplementary portion. An exemplary supplementary portion may include vitamins necessary for mitochondrion functions. An exemplary pain-relief medicine may also heal sleep disorder, menstrual problems, digestive problems, mental problems, hormonal problems, increased brain pressure, and combinations thereof. An exemplary pain-relief medicine may be used with doctor's prescription.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, subject matter of the present disclosure may lie in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

According to some embodiments, a pain-relief medicine for healing headache is provided. The pain-relief medicine for healing headache includes an herbal portion and a supplementary portion. The herbal portion includes pimpinella anisum powder with a weight percentage in a range of 4 wt. % to 50 wt. % of a total amount of the herbal portion; artemisia vulgaris powder with a weight ratio to the pimpinella anisum powder in a range of 7:50 to 15:1 (artemisia vulgaris powder:pimpinella anisum powder); viola odorata powder with a weight ratio to the pimpinella anisum powder in a range of 2:25 to 25:2 (viola odorata powder:pimpinella anisum powder); Cyperus species powder with a weight ratio to the pimpinella anisum powder in a range of 1:50 to 15:2 (Cyperus species powder:pimpinella anisum powder); matricaria chamomilla powder with a weight ratio to the pimpinella anisum powder in a range of 1:50 to 15:4 (matricaria chamomilla powder:pimpinella anisum powder); salvia officinalis powder with a weight ratio to the pimpinella anisum powder in a range of 1:50 to 25:4 (salvia officinalis powder:pimpinella anisum powder); aesculus hippocastanum powder with a weight ratio to the pimpinella anisum powder in a range of 1:25 to 15:2 (aesculus hippocastanum powder:impinella anisum powder); cymbopogon citratus powder with a weight ratio to the pimpinella anisum powder in a range of 1:25 to 5:1 (cymbopogon citratus powder:pimpinella anisum powder); trachyspermum ammi powder with a weight ratio to the pimpinella anisum powder in a range of 1:50 to 5:1 (trachyspermum ammi powder:pimpinella anisum powder); and curcuma longa powder with a weight ratio to the pimpinella anisum powder in a range of 1:50 to 5:1 (curcuma longa powder:pimpinella anisum powder). The supplementary portion with a weight ratio to the herbal portion in a range of 1:7 to 7:1 (herbal portion:supplementary portion) includes a coenzyme Q₁₀ powder with a weight ratio to the pimpinella anisum powder in a range of 50:1 to 1:10 (coenzyme Q₁₀:pimpinella anisum powder); carnitine powder with a weight ratio to the pimpinella anisum powder in a range of 75:1 to 3:5 (carnitine:pimpinella anisum powder); magnesium powder with a weight ratio to the pimpinella anisum powder in a range of 25:1 to 1:10 (magnesium:pimpinella anisum powder); α-lipoic acid powder with a weight ratio to the pimpinella anisum powder in a range of 75:1 to 1:5 (α-lipoic acid:pimpinella anisum powder); zinc powder with a weight ratio to the pimpinella anisum powder in a range of 10:1 to 1:50 (zinc:pimpinella anisum powder); iron powder with a weight ratio to the pimpinella anisum powder in a range of 10:1 to 1:25 (iron:pimpinella anisum powder); vitamin C powder with a weight ratio to the pimpinella anisum powder in a range of 125:1 to 1:5 (vitamin C:pimpinella anisum powder); vitamin E powder with a weight ratio to the pimpinella anisum powder in a range of 90:4 to 4.5:50 (vitamin E:pimpinella anisum powder); vitamin D powder with a weight ratio to the pimpinella anisum powder in a range of 0.0125:4 to 0.00125:50 (vitamin D:pimpinella anisum powder); vitamin B powder with a weight ratio to the pimpinella anisum powder in a range of 2.5:1 to 1:500 (vitamin B:pimpinella anisum powder); and caffeine powder with a weight ratio to the pimpinella anisum powder in a range of 25:1 to 1:50 (caffeine:pimpinella anisum powder).

According to some embodiments, the pain-relief medicine for healing headache includes at least one adhesive substance with a weight percentage of a total amount of the pain-relief medicine in a range of 2 wt. % to 10 wt. %.

According to some embodiments, the adhesive substance includes starch and/or polyvinylpyrrolidone (PVP).

According to some embodiments, the vitamin B includes thiamin (B1), riboflavin (B2), niacin (B3), pantothenic acid (B5), pyridoxine (B6), biotin (B7), folate [folic acid] (B9) and/or cobalamin (B12).

According to some embodiments, each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder includes powder of a root, a stem, a leaf, a flower, a fruit pimpinella anisum, artemisia vulgaris, viola odorata, Cyperus species, matricaria chamomilla, salvia officinalis, aesculus hippocastanum, cymbopogon citratus, trachyspermum ammi, and curcuma longa.

According to some embodiments, powder particles of each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder includes particles with an average particle size in a range of 10 micrometer (μm) to 200 μm.

According to some embodiments, the pain-relief medicine for

healing headache has a form of a tablet, a capsule, s syrup, a tea bag, suppository and/or effervescent tablet.

According to some embodiments, powder particles of each of the coenzyme Q₁₀ powder, the carnitine powder, the magnesium powder, the α-lipoic acid powder, the zinc powder, the iron powder, the vitamin C powder, the vitamin E powder, the vitamin D powder, the vitamin B powder, and the caffeine powder includes particles with an average particle size in a range of 10 micrometer (μm) to 200 μm.

According to some embodiments, the Cyperus species powder includes powder of of Cyperus rotundus, Cyperus javanicus, Cyperus brevifolius, Cyperus kyllingia, Cyperus monocephalus, Cyperus compressus, Cyperus articulatus, Cyperus pedunculatus, Cyperus nitidus, Cyperus sexangularis, Cyperus kilimandscharicus, Cyperus latifolius, Cyperus maculatus, Cyperus natalensis, Cyperus erectus, Cyperus mundii, Cyperus esculentus and/or Cyperus flavescens.

According to some embodiments, a method for producing a pain-relief medicine is provided. The method includes: forming an herbal portion; forming a supplementary portion by mixing powders of a plurality of supplementary compounds, the plurality of supplementary compounds comprising at least one of coenzyme Q₁₀ powder, carnitine powder, magnesium powder, α-lipoic acid powder, zinc powder, iron powder, vitamin C powder, vitamin E powder, vitamin D powder, vitamin B powder, caffeine powder, and combinations thereof; and forming an herbal-supplementary mixture by mixing the herbal portion and the supplementary portion. Forming the herbal portion includes forming an herbal mixture by mixing powders of a plurality of herbal compounds, the powders of the plurality of herbal compounds comprising artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder; forming an herbal suspension by mixing the herbal mixture with a hydroalcoholic solution, the hydroalcoholic solution comprising an alcohol portion and a water portion; and isolating an aqueous-herbal extract of the herbal mixture by evaporating the alcohol portion of the herbal suspension.

According to some embodiments, the method for producing a pain-relief medicine, further comprising forming the powders of the plurality of the supplementary compounds by grinding each respective supplementary compound to particles, each particle with an average particle size in a range of 1 micrometer (μm) to 200 μm.

According to some embodiments, forming the herbal suspension comprises mixing the herbal mixture with the hydroalcoholic solution using a mixer with a stirring speed in a range of 50 revolutions per minute (rpm) to 200 rpm.

According to some embodiments, forming the herbal suspension comprises mixing the herbal mixture with the hydroalcoholic solution using a mixer for a time period in a range of 5 minutes to 10 minutes.

According to some embodiments, forming the herbal-supplementary mixture comprises mixing the herbal portion and the supplementary portion in a mixer with a stirring speed in a range of 50 revolutions per minute (rpm) to 200 rpm.

According to some embodiments, forming the herbal-supplementary mixture comprises mixing the herbal portion and the supplementary portion in a mixer for a time period in a range of 5 minutes to 10 minutes.

According to some embodiments, the method for producing a pain-relief medicine, including: forming a tablet mixture by mixing the herbal portion with the supplementary portion; and applying a pressure on the tablet mixture in a range of 2500 kilopascal (kPa) to 6500 kPa.

According to some embodiments, a pain-relief medicine is provided. The pain-relief medicine including: an herbal portion, wherein the herbal portion includes: a pimpinella anisum powder with a weight percentage in a range of about 1 wt. % to about 100 wt. % of a total amount of the herbal portion; an artemisia vulgaris powder with a weight percentage in a range of about 1 wt. % to about 90 wt. % of a total amount of the herbal portion; a viola odorata powder with a weight percentage in a range of about 1 wt. % to about 90 wt. % of a total amount of the herbal portion; a Cyperus species powder with a weight percentage in a range of about 1 wt. % to about 50 wt. % of a total amount of the herbal portion; a matricaria chamomilla powder with a weight percentage in a range of about 1 wt. % to about 50 wt. % of a total amount of the herbal portion; a salvia officinalis powder with a weight percentage in a range of about 1 wt. % to about 50 wt. % of a total amount of the herbal portion; an aesculus hippocastanum powder with a weight percentage in a range of about 1 wt. % to about 50 wt. % of a total amount of the herbal portion; a cymbopogon citratus powder with a weight percentage in a range of about 1 wt. % to about 50 wt. % of a total amount of the herbal portion; a trachyspermum ammi powder with a weight percentage in a range of about 1 wt. % to about 50 wt. % of a total amount of the herbal portion; and/or a curcuma longa powder with a weight percentage in a range of about 1 wt. % to about 50 wt. % of a total amount of the herbal portion; and a supplementary portion, wherein the supplementary portion includes at least two of: coenzyme Q₁₀ powder with a weight percentage in a range of about 1 wt. % to about 90 wt. % of a total amount of the supplementary portion; a carnitine powder with a weight percentage in a range of about 1 wt. % to about 100 wt. % of a total amount of the supplementary portion; a magnesium powder with a weight percentage in a range of about 1 wt. % to about 90 wt. % of a total amount of the supplementary portion; an α-lipoic acid powder with a weight percentage in a range of about 1 wt. % to about 40 wt. % of a total amount of the supplementary portion; a zinc powder with a weight percentage in a range of about 1 wt. % to about 40 wt. % of a total amount of the supplementary portion; an iron powder with a weight percentage in a range of about 1 wt. % to about 40 wt. % of a total amount of the supplementary portion; a vitamin C powder with a weight percentage in a range of about 1 wt. % to about 40 wt. % of a total amount of the supplementary portion; a vitamin E powder with a weight percentage in a range of about 1 wt. % to about 40 wt. % of a total amount of the supplementary portion; a vitamin D powder with a weight percentage in a range of about 1 wt. % to about 40 wt. % of a total amount of the supplementary portion; a vitamin B powder with a weight percentage in a range of about 1 wt. % to about 40 wt. % of a total amount of the supplementary portion; or a caffeine powder with a weight percentage in a range of about 1 wt. % to about 40 wt. % of a total amount of the supplementary portion.

According to some embodiments, a ratio of the herbal portion to the supplementary portion by weight is between about 1:7 to about 7:1; and/or a ratio of the herbal portion to the supplementary portion by weight is between about 3:5 to about 5:3.

According to some embodiments, each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder comprises powder of at least one of root, stem, leaf, flower or fruit of the respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa; and/or powder particles of each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder comprises particles with an average particle size in a range of about 10 micrometer (μm) to about 200 μm.

According to some embodiments, the pain-relief medicine has a form of a tablet; a capsule; a syrup; a tea bag; a suppository; and/or an effervescent tablet.

It may be appreciated that one, some or all of the pain-relief medicines of the present disclosure may be used for treating one or more other illnesses and/or conditions in addition to and/or as an alternative to treating headaches.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Various operations of embodiments and/or examples are provided herein. The order in which some or all of the operations are described herein should not be construed as to imply that these operations are necessarily order dependent. Alternative ordering will be appreciated by one skilled in the art having the benefit of this description. Further, it will be understood that not all operations are necessarily present in each embodiment and/or example provided herein. Also, it will be understood that not all operations are necessary in some embodiments and/or examples.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. A pain-relief medicine for healing headache, comprising:
an herbal portion, comprising:
pimpinella anisum powder with a weight percentage in a range of 4 wt. % to 50 wt. % of a total amount of the herbal portion;
artemisia vulgaris powder with a weight ratio to the pimpinella anisum powder in a range of 7:50 to 15:1 (artemisia vulgaris powder:pimpinella anisum powder);
viola odorata powder with a weight ratio to the pimpinella anisum powder in a range of 2:25 to 25:2 (viola odorata powder:pimpinella anisum powder);
Cyperus species powder with a weight ratio to the pimpinella anisum powder in a range of 1:50 to 15:2 (Cyperus species powder:pimpinella anisum powder);
matricaria chamomilla powder with a weight ratio to the pimpinella anisum powder in a range of 1:50 to 15:4 (matricaria chamomilla powder:pimpinella anisum powder);
salvia officinalis powder with a weight ratio to the pimpinella anisum powder in a range of 1:50 to 25:4 (salvia officinalis powder:pimpinella anisum powder);
aesculus hippocastanum powder with a weight ratio to the pimpinella anisum powder in a range of 1:25 to 15:2 (aesculus hippocastanum powder:pimpinella anisum powder);
cymbopogon citratus powder with a weight ratio to the pimpinella anisum powder in a range of 1:25 to 5:1 (cymbopogon citratus powder:pimpinella anisum powder);
trachyspermum ammi powder with a weight ratio to the pimpinella anisum powder in a range of 1:50 to 5:1 (trachyspermum ammi powder:pimpinella anisum powder); and
curcuma longa powder with a weight ratio to the pimpinella anisum powder in a range of 1:50 to 5:1 (curcuma longa powder:pimpinella anisum powder); and
a supplementary portion with a weight ratio to the herbal portion in a range of 1:7 to 7:1 (herbal portion:supplementary portion), the supplementary portion comprising:
coenzyme Q₁₀ powder with a weight ratio to the pimpinella anisum powder in a range of 50:1 to 1:10 (coenzyme Q₁₀:pimpinella anisum powder);
carnitine powder powder with a weight ratio to the pimpinella anisum in a range of 75:1 to 3:5 (carnitine:pimpinella anisum powder);
magnesium powder with a weight ratio to the pimpinella anisum powder in a range of 25:1 to 1:10 (magnesium:pimpinella anisum powder);
α-lipoic acid powder with a weight ratio to the pimpinella anisum powder in a range of 75:1 to 1:5 (α-lipoic acid:pimpinella anisum powder);
zinc powder with a weight ratio to the pimpinella anisum powder in a range of 10:1 to 1:50 (zinc:pimpinella anisum powder);
iron powder with a weight ratio to the pimpinella anisum powder in a range of 10:1 to 1:25 (iron:pimpinella anisum powder);
vitamin C powder with a weight ratio to the pimpinella anisum powder in a range of 125:1 to 1:5 (vitamin C:pimpinella anisum powder);
vitamin E powder with a weight ratio to the pimpinella anisum powder in a range of 90:4 to 4.5:50 (vitamin E:pimpinella anisum powder);
vitamin D powder with a weight ratio to the pimpinella anisum powder in a range of 0.0125:4 to 0.00125:50 (vitamin D:pimpinella anisum powder);
vitamin B powder with a weight ratio to the pimpinella anisum powder in a range of 2.5:1 to 1:500 (vitamin B:pimpinella anisum powder); and
caffeine powder with a weight ratio to the pimpinella anisum powder in a range of 25:1 to 1:50 (caffeine:pimpinella anisum powder).

2. The pain-relief medicine of claim 1, further comprising at least one adhesive substance with a weight percentage of a total amount of the pain-relief medicine in a range of 2 wt. % to 10 wt. %.

3. The pain-relief medicine of claim 2, wherein the adhesive substance comprises at least one of starch, polyvinylpyrrolidone (PVP), and combinations thereof.

4. The pain-relief medicine of claim 1, wherein the vitamin B comprises at least one of thiamin (B1), riboflavin (B2), niacin (B3), pantothenic acid (B5), pyridoxine (B6), biotin (B7), folate [folic acid] (B9), cobalamin (B12), and combinations thereof.

5. The pain-relief medicine of claim 1, wherein each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder comprises powder of at least one of root, stem, leaf, flower, fruit, and combinations thereof of the respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa.

6. The pain-relief medicine of claim 1, wherein powder particles of each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder comprises particles with an average particle size in a range of 10 micrometer (μm) to 200 μm.

7. The pain-relief medicine of claim 1, wherein the pain-relief medicine has a form of at least one of tablet, capsule, syrup, tea bag, suppository, effervescent tablet, and combinations thereof.

8. The pain-relief medicine of claim 1, wherein powder particles of each of the coenzyme Q₁₀ powder, the carnitine powder, the magnesium powder, the α-lipoic acid powder, the zinc powder, the iron powder, the vitamin C powder, the vitamin E powder, the vitamin D powder, the vitamin B powder, and the caffeine powder comprises particles with an average particle size in a range of 10 micrometer (μm) to 200 μm.

9. The pain-relief medicine of claim 1, wherein the Cyperus species powder comprises powder of at least one of Cyperus rotundus, Cyperus javanicus, Cyperus brevifolius, Cyperus kyllingia, Cyperus monocephalus, Cyperus compressus, Cyperus articulatus, Cyperus pedunculatus, Cyperus nitidus, Cyperus sexangularis, Cyperus kilimandscharicus, Cyperus latifolius, Cyperus maculatus, Cyperus natalensis, Cyperus erectus, Cyperus mundii, Cyperus esculentus, Cyperus flavescens, and combinations thereof.

10. A method for producing a pain-relief medicine, the method comprising:
forming an herbal portion, comprising:
forming an herbal mixture by mixing powders of a plurality of herbal compounds, the powders of the plurality of herbal compounds comprising artemisia vulgaris powder, viola odorata powder, Cyperus species powder, matricaria chamomilla powder, salvia officinalis powder, aesculus hippocastanum powder, cymbopogon citratus powder, trachyspermum ammi powder, and curcuma longa powder;
forming an herbal suspension by mixing the herbal mixture with a hydroalcoholic solution, the hydroalcoholic solution comprising an alcohol portion and a water portion; and
isolating an aqueous-herbal extract of the herbal mixture by evaporating the alcohol portion of the herbal suspension;
forming a supplementary portion by mixing powders of a plurality of supplementary compounds, the plurality of supplementary compounds comprising at least one of coenzyme Q₁₀ powder, carnitine powder, magnesium powder, α-lipoic acid powder, zinc powder, iron powder, vitamin C powder, vitamin E powder, vitamin D powder, vitamin B powder, caffeine powder, and combinations thereof; and
forming an herbal-supplementary mixture by mixing the herbal portion and the supplementary portion.

11. The method of claim 10, further comprising forming the powders of the plurality of the supplementary compounds by grinding each respective supplementary compound to particles, each particle with an average particle size in a range of 1 micrometer (μm) to 200 μm.

12. The method of claim 10, wherein forming the herbal suspension comprises mixing the herbal mixture with the hydroalcoholic solution using a mixer with a stirring speed in a range of 50 revolutions per minute (rpm) to 200 rpm.

13. The method of claim 10, wherein forming the herbal suspension comprises mixing the herbal mixture with the hydroalcoholic solution using a mixer for a time period in a range of 5 minutes to 10 minutes.

14. The method of claim 10, wherein forming the herbal-supplementary mixture comprises mixing the herbal portion and the supplementary portion in a mixer with a stirring speed in a range of 50 revolutions per minute (rpm) to 200 rpm.

15. The method of claim 10, wherein forming the herbal-supplementary mixture comprises mixing the herbal portion and the supplementary portion in a mixer for a time period in a range of 5 minutes to 10 minutes.

16. The method of claim 10, comprising:
forming a tablet mixture by mixing the herbal portion with the supplementary portion; and
applying a pressure on the tablet mixture in a range of 2500 kilopascal (kPa) to 6500 kPa.

17. A pain-relief medicine, comprising:
an herbal portion, wherein the herbal portion comprises at least one of:
a pimpinella anisum powder with a weight percentage in a range of about 1 wt. % to about 100 wt. % of a total amount of the herbal portion;
an artemisia vulgaris powder with a weight percentage in a range of about 1 wt. % to about 90 wt. % of a total amount of the herbal portion;
a viola odorata powder with a weight percentage in a range of about 1 wt. % to about 90 wt. % of a total amount of the herbal portion;
a Cyperus species powder with a weight percentage in a range of about 1 wt. % to about 50 wt. % of a total amount of the herbal portion;
a matricaria chamomilla powder with a weight percentage in a range of about 1 wt. % to about 50 wt. % of a total amount of the herbal portion;
a salvia officinalis powder with a weight percentage in a range of about 1 wt. % to about 50 wt. % of a total amount of the herbal portion;
an aesculus hippocastanum powder with a weight percentage in a range of about 1 wt. % to about 50 wt. % of a total amount of the herbal portion;
a cymbopogon citratus powder with a weight percentage in a range of about 1 wt. % to about 50 wt. % of a total amount of the herbal portion;
a trachyspermum ammi powder with a weight percentage in a range of about 1 wt. % to about 50 wt. % of a total amount of the herbal portion; or
a curcuma longa powder with a weight percentage in a range of about 1 wt. % to about 50 wt. % of a total amount of the herbal portion; and
a supplementary portion, wherein the supplementary portion comprises at least two of:
a coenzyme Q₁₀ powder with a weight percentage in a range of about 1 wt. % to about 90 wt. % of a total amount of the supplementary portion;
a carnitine powder with a weight percentage in a range of about 1 wt. % to about 100 wt. % of a total amount of the supplementary portion;
a magnesium powder with a weight percentage in a range of about 1 wt. % to about 90 wt. % of a total amount of the supplementary portion;
an α-lipoic acid powder with a weight percentage in a range of about 1 wt. % to about 40 wt. % of a total amount of the supplementary portion;
a zinc powder with a weight percentage in a range of about 1 wt. % to about 40 wt. % of a total amount of the supplementary portion;
an iron powder with a weight percentage in a range of about 1 wt. % to about 40 wt. % of a total amount of the supplementary portion;
a vitamin C powder with a weight percentage in a range of about 1 wt. % to about 40 wt. % of a total amount of the supplementary portion;
a vitamin E powder with a weight percentage in a range of about 1 wt. % to about 40 wt. % of a total amount of the supplementary portion;
a vitamin D powder with a weight percentage in a range of about 1 wt. % to about 40 wt. % of a total amount of the supplementary portion;
a vitamin B powder with a weight percentage in a range of about 1 wt. % to about 40 wt. % of a total amount of the supplementary portion; or
a caffeine powder with a weight percentage in a range of about 1 wt. % to about 40 wt. % of a total amount of the supplementary portion.

18. The pain-relief medicine of claim 17, wherein at least one of:
a ratio of the herbal portion to the supplementary portion by weight is between about 1:7 to about 7:1; or
a ratio of the herbal portion to the supplementary portion by weight is between about 3:5 to about 5:3.

19. The pain-relief medicine of claim 17, wherein at least one of:
each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder comprises powder of at least one of root, stem, leaf, flower or fruit of the respective pimpinella anisum, artemisia vulgaris, viola odorata, Cyperus species, matricaria chamomilla, salvia officinalis, aesculus hippocastanum, cymbopogon citratus, trachyspermum ammi, and curcuma longa; or
powder particles of each of the pimpinella anisum powder, the artemisia vulgaris powder, the viola odorata powder, the Cyperus species powder, the matricaria chamomilla powder, the salvia officinalis powder, the aesculus hippocastanum powder, the cymbopogon citratus powder, the trachyspermum ammi powder, and the curcuma longa powder comprises particles with an average particle size in a range of about 10 micrometer (μm) to about 200 μm.

20. The pain-relief medicine of claim 17, wherein the pain-relief medicine has a form of at least one of:
a tablet;
a capsule;
a syrup;
a tea bag;
a suppository; or
an effervescent tablet.
